(19) United States
(12) Patent Application Publication
(10) Pub. No.: US 2016/0031955 A1
(43) Pub. Date: Feb. 4, 2016

(54) Methods for Treating Mitochondrial Disorders and Neurodegenerative Disorders

(71) Applicants: INSERM (INSTITUT NATIONAL DE LA SANTÉ ET DE LA RECHERCHE MÉDICALE, Paris (FR); Institut Gustave Rouddy, Villejuif (FR); Universite Paris-Sud, Orsay (FR)

(72) Inventors: Nazanine Modjtahedi, Villejuif (FR); Guido Kroemer, Villejuif (FR)

(21) Appl. No.: 14/762,610

(22) PCT Filed: Jan. 30, 2014
(86) PCT No.: PCT/EP2014/051842

§ 371 Date: Jul. 22, 2015

(30) Foreign Application Priority Data
Jan. 31, 2013 (EP) 13305112.8

(51) Int. Cl. C07K 14/47; C12Q 1/68

(52) U.S. Cl. CPC C07K 14/47; C12Q 1/6883; A61K 38/00

(57) ABSTRACT

The present invention relates to a recombinant inner mitochondrial membrane polypeptide, and its use in methods for treating mitochondrial disorders.

### FIELD OF THE INVENTION

The present invention provides methods for treating mitochondrial disorders and neurodegenerative diseases using recombinant proteins or gene therapy.

### BACKGROUND OF THE INVENTION

Mitochondria, found in most eukaryotic cells, control cellular processes such as proliferation, differentiation and death through the generation of energy and the regulation of many important metabolic signaling pathways.

The Mitochondrial dysfunction has not only been associated to aging-related pathologies but also to human diseases that are gathered under the name of “mitochondrial disorders”. These disorders are a group of heterogeneous diseases caused by the dysfunction of the most important energy-producing metabolic machinery, which is the mitochondrial respiratory chain (RC). This chain is composed of five multi-protein complexes that are embedded in the inner mitochondrial membrane and allow the conversion of the food molecules energy into ATP, which is the source of energy for all living cells.

The mitochondrial RC is the only cellular machinery to be controlled both by mitochondrial and nuclear genes and for this reason, a large number of mutations found in both genomes have been associated to mitochondrial disorders. During cell division, mitochondria are distributed randomly in the daughter cells and their number varies from one cell to another. In different cell types, the number of mitochondrial DNA (mtDNA) may range from several hundred to several thousand and it should be noted that in a normal cell, all mtDNA molecules are identical. Mutations targeting the mtDNA are only found in a fraction of the molecules present in the cell and, for this reason, the clinical expression of pathogenic mutations depends on the relative proportion of mutated and normal mtDNA. In conclusion, a minimum number of mutated molecules is required for the induction of the mitochondrial dysfunction. On the contrary, the mutations in nuclear genes that encode for mitochondrial proteins follow the rules of Mendelian inheritance. Mitochondrial disorders are very heterogeneous. Subclasses of these diseases that associate muscular symptoms with brain involvement are often called mitochondrial encephalomyopathies.

Symptoms can include poor growth, loss of muscle coordination, muscle weakness, visual problems, hearing problems, learning disabilities, heart disease, liver disease, kidney disease, gastrointestinal disorders, respiratory disorders, neurological problems, autonomic dysfunction, and dementia.

The severity of the specific mitochondrial defect may also be great or small. Some minor defects cause only “exercise intolerance”, with no serious illness or disability. Some defects could affect multiple tissues leading consequently to multi-system disorders.

Although mitochondrial diseases vary greatly in their expression from person to person, several major clinical categories of these conditions have been defined, based on the most common phenotypic features, symptoms, and signs associated with the particular mutations that tend to cause them.

Mitochondrial diseases could be provoked by inherited or acquired mutations. About 1 in 4,000 children in the United States will develop mitochondrial disease by the age of 10 years. Up to 4,000 children per year in the US are born with a type of mitochondrial disease. While some of the affected children live fairly normal lives, some others that are severely affected could succumb to the disease before adolescence.

Adults can also be diagnosed with late onset mitochondrial diseases and be affected in a manner similar to affected children.

Mitochondrial defects have also been linked to the most common diseases of aging that include Alzheimer disease, Parkinson, cardiovascular disease and also cancer or type 2 diabetes.

Mitochondria are composed of an outer mitochondrial membrane, an intermembrane space, an inner mitochondrial membrane and a matrix. The multi-protein complexes that compose the RC are embedded in the inner mitochondrial membrane and correspond to enzymatic series of electron donors and acceptors. Each electron donor passes electrons to a more electronegative acceptor, which in turn donates these electrons to another acceptor, a process that continues down the series until electrons are passed to oxygen, the most electronegative and terminal electron acceptor in the chain. The electron transfer from complex I to complex IV and this generates a proton gradient, which is used by the complex V to synthesize ATP. The entire process is called oxidative phosphorylation, since ADP is phosphorylated to ATP. Beside the RC complex II that contains only protein subunits encoded by the mitochondrial genome, the other four complexes (complex I, III, IV and V) are composed of subunits encoded by both mitochondrial and nuclear genes. The optimal functioning of the RC depends on the correct biogenesis, import and assembly of each multi-protein complex.

One of the mitochondrial factors that have been described to regulate the RC activity by participating to the assembly or the stability of the complexes is the nuclear-encoded protein AIF.

Apoptosis inducing factor (AIF) was initially characterized as a redox-active flavoprotein that is contained in the intermembrane space of mitochondria from healthy cells, yet translocates to the cytosol and the nucleus upon the apoptosis-associated induction of mitochondrial outer membrane permeabilization (MOMP). AIF has been involved in the regulation and execution of apoptosis throughout eukaryote phylogeny, in mammals, flies, nematodes, and yeast. In the cytosol, AIF can signal for phosphatidylserine exposure on the plasma membrane. Moreover, in the nucleus, AIF can participate in chromatin condensation and caspase-independent large-scale DNA fragmentation, likely through direct electrostatic interaction with the DNA phosphodiester backbone, as well as with other proteins that possess latent DNase activities. AIF-deficient cells are resistant against a restricted panel of cell death inducers. In particular, mouse embryonic stem (ES) cells lacking AIF due to homologous recombination (Aif−/y) fail to undergo cavitation, a process of cell loss within the early developing embryo that reflects the first wave of programmed cell death during ontogeny.

Beyond its role in apoptosis, AIF also participates in normal mitochondrial metabolism. Indeed, the absence of AIF causes a respiratory chain defect in all species investigated (Hangen et al., 2010a; Vahsen et al., 2004; Joza et al., 2008; Wissing et al., 2004; Pospisilik et al., 2007). Mice affected by a hypomorphic AIF mutation (“Harlequin mice”) manifest a respiratory chain deficiency coupled to the post-transcriptional downregulation of protein subunits belonging to respiratory chain complexes I, III and IV (Vahsen et al., 2004; Posposilik et al., 2007). A similar phenotype can be reproduced by tissue-specific knockout of AIF (Joza et al., 2005), and is observed in human infants with loss-of-function mutations of AIF (Ghezzi et al., 2010). Both Harlequin mice and AIF-deficient infants similarly develop a severe neuromuscular mitochondriopathy leading to premature death (Ghezzi et al., 2012; Klein et al., 2002).

The mechanisms through which AIF affects the assembly or stability of respiratory chain complexes are unknown and to date, no successful therapy has been developed.

Thus, there is still an unmet need in the art for effective therapies for treating mitochondrial disorders, in particular complex I disorders.

### SUMMARY OF THE INVENTION

The inventors have discovered that, in normal mitochondria, AIF allows the stabilization of another mitochondrial protein, known as MIA40, through direct protein-protein interaction, and that MIA40 acts downstream of AIF to ensure optimal abundance and function of the respiratory chain supercomplex.

The inventors have developed a recombinant MIA40, which is directly targeted to the inner mitochondrial membrane and bypasses the necessity for AIF.

Thus, in one aspect, the present invention relates to a polypeptide comprising:
a) a mitochondrial inner membrane localization signal and
b) the amino acid sequence as set forth in SEQ ID NO:1 or a variant thereof having at least 80% identity with SEQ ID NO:1.

The amino acid sequence set forth in SEQ ID NO:1 corresponds to amino acids 28 to 142 of the MIA40 protein isoform 1. It encompasses the oxidase motif and the CX9C—CX9C domain, both of which were shown to play a role in MIA40's function.

In another aspect, the invention relates to a nucleic acid encoding said polypeptide.

In yet another aspect, the invention relates to a polypeptide or nucleic acid as defined above for use in therapy.

Finally, the invention also relates to a method for diagnosing a mitochondrial disorder in a patient comprising the step of analyzing the gene encoding MIA40 in a biological sample obtained from said patient.

### DETAILED DESCRIPTION OF THE INVENTION

Polypeptides According to the Invention

In one aspect, the invention relates to a polypeptide comprising:

a) a mitochondrial inner membrane localization signal
and
b) the amino acid sequence as set forth in SEQ ID NO:1 or a variant thereof having at least 80% identity with SEQ ID NO:1.

In other terms, the polypeptide according to the present invention is a recombinant MIA40 comprising a mitochondrial inner membrane localization signal.

As explained above, the amino acid sequence set forth in SEQ ID NO:1 corresponds to corresponds to amino acids 28 to 142 of the MIA40 protein iso form 1. This sequence is present in both MIA40.1 and MIA40.2 isoforms.

As used herein, the term “MIA40” has its general meaning in the art. It refers to any isoform of MIA40 protein, from any mammalian species. MIA40 is also known as CHCHD4.

In humans, MIA40 is encoded by the gene referenced under Genbank accession number Gene ID: 131474. This gene encodes 2 splice variants of MIA40:
MIA40.1 (or MIA40 isoform 1) is the predominant isoform. It is encoded by exons 1, 3 and 4 of the MIA40 gene and has the sequence as set forth in SEQ ID NO: 2, also known as Genbank accession number NP_{—}001091972.1
MIA40.2 (or MIA40 isoform 2) is encoded by exons 1, 2, 3 and 4 of the MIA40 gene and has the sequence as set forth in SEQ ID NO: 3, also known as Genbank accession number NP_{—}653237.1.

MIA40 is a well conserved protein among mammalian species.

Indeed, the percentage of identity between the human MIA40 iso form 1 and MIA40 isoform 1 of other species is as follows:
Pan troglotydes (chimpanzee, primate): 99.3%
Rattus norvegicus (rat, rodent): 83.9%
Mus musculus (mouse, rodent): 83.2%
Bos taurus (cow, bovine): 80.7%

The percentage of identity can be calculated according to standard methods in the art. The two sequences to be compared are aligned and the number of identical residues is calculated and divided by the total number of residues aligned.

Typically, the percentage of identity between two polypeptides can be calculated by software based on Lipman-Pearson protein alignment.

In one embodiment, the polypeptide according to the present invention comprises a sequence having at least 80% identity with the amino acid sequence as set forth in SEQ ID NO:1.

In a preferred embodiment, the polypeptide according to the invention has at least 85%, preferably at least 90%, even more preferably at least 91%, 92%, 93%, 94%, 95%, 96%, 97%, 98%, 99%, 99.5%, 99.8%, 99.9% identity with the amino acid sequence as set forth in SEQ ID NO:1.

Typically, the recombinant MIA40 protein of the present invention comprises a sequence having at least 80%, preferably at least 85%, even more preferably at least 90%, 91%, 92%, 93%, 94%, 95%, 96%, 97%, 98%, 99%, 99.5%, 99.8%, 99.9% identity with the amino acid sequence as set forth in SEQ ID NO: 2, wherein SEQ ID NO: 2 corresponds to the human MIA40 isoform 1.

Typically, the recombinant MIA40 protein of the present invention comprises a sequence having at least 80%, preferably at least 85%, even more preferably at least 90%, 91%, 92%, 93%, 94%, 95%, 96%, 97%, 98%, 99%, 99.5%, 99.8%, 99.9% identity with the amino acid sequence as set forth in SEQ ID NO: 3, wherein SEQ ID NO: 3 corresponds to the human MIA40 isoform 2.

In one embodiment, the recombinant MIA40 protein of the invention comprises at least 4 cysteine residues, wherein said 4 cysteine residues correspond to amino acids 64, 74, 87 and 97 of the MIA40 iso form 1 (as set forth in SEQ ID NO: 2). These cysteine residues have been shown to play a role in the normal function of MIA40.

In another embodiment, the recombinant MIA40 protein of the invention comprises at least 6 cysteine residues, wherein said 6 cysteine residues correspond to amino acids 53 55, 64, 74, 87 and 97 of the MIA40 isoform 1 (as set forth in SEQ ID NO: 2).

The cysteines located at positions 53 and 55 are part of the oxidase motif of MIA40 necessary for MIA40's function.

Typically, the recombinant MIA40 protein of the present invention is capable of restoring the levels of respiratory chain complex I subunits in AIF-depleted cells. Typically, the capacity of a recombinant protein to restore normal function in AIF-depleted cells can be measured according to the following test:

4 days after knocking-down AIF with the siRNA, confluent U2OS cells overexpressing molecules having the sequences as set forth in SEQ ID NO: 12 and SEQ ID NO: 13 are lysed in 1% SDS and then the proteins present in lysate are quantified (Bio-Rad DC protein assay) and subjected to direct Western blot analyses and the levels of respiratory chain complexes subunits assessed using specific antibodies and compared to control levels.

As used herein, the expression “mitochondrial inner membrane localization signal” (MLS) refers to an amino acid sequence, which targets a given polypeptide to the inner mitochondrial membrane and allows the functional part of the recombinant protein to face the intermembrane space.

Typically, the MLS 1) allows the import of the recombinant protein to the mitochondrion, 2) targets it to the inner membrane of the organelle and 3) allows the functional part of the recombinant protein to face the intermembrane space (Schmidt, et al., 2010).

It is well established that many nuclear-encoded proteins that are inserted in the inner mitochondrial membrane and function in the intermembrane space of the organelle, are synthesized in the cytoplasm as precursor proteins that possess a bipartite N-terminal MLS (Claros et al. 1996; Claros et al., 1997 Herrmann et al. 2005; Schmidt et al., 2010). Briefly, the N-terminal part of the MLS consists of a stretch of 15-50 positively charged amino acids that direct the protein through the outer and inner mitochondrial membrane import machinery translocases, in a sequential manner, before being cleaved-off by mitochondrial peptidases such as the mitochondrial processing peptidase (MPP) localized in the matrix. The second half of the MLS consists of a stretch of hydrophobic amino acids that allows the anchorage of the N-terminus of the protein in the inner membrane and the sorting of the c-terminal functional part of the protein in the intermembrane space (Schmidt et al. 2010).

In one embodiment, the mitochondrial inner membrane localization signal (MLS) has the amino acid sequence selected from the group consisting of SEQ ID NO: 4, SEQ ID NO: 5, SEQ ID NO: 25 and a variant thereof having at least 90% identity with SEQ ID NO: 4, SEQ ID NO: 5 or SEQ ID NO: 25.

In a preferred embodiment, the amino acid sequence of MLS according to the invention has at least 90%, even more preferably at least 91%, 92%, 93%, 94%, 95%, 96%, 97%, 98%, 99%, 99.5%, 99.8%, 99.9% identity with the amino acid sequence selected from the group consisting of SEQ ID NO: 4, SEQ ID NO: 5 and SEQ ID NO: 25.

In a preferred embodiment, the mitochondrial inner membrane localization signal according to the invention can be the mitochondrial inner membrane localization signal of the AIF protein (Susin et al., 1999; Otera, et al. 2005; Herrmann et al., 2005). This mitochondrial inner membrane localization signal has the sequence set forth in SEQ ID NO: 4, which corresponds to residues 1 to 120 of the human AIF protein. It has been experimentally proved that the above-mentioned segment of AIF includes a typical bipartite MLS (Otera et al. 2005) composed of an N-terminal mitochondrial addressing signal of 52 residues long, an MPP cleavage site that allows the maturation of the protein through the cleavage of the precursor at the position 53 and finally a cluster of hydrophobic residues, between positions 66 and 84, required for the correct sorting of the protein to the IMS and its anchorage to the inner membrane.

In another embodiment, the mitochondrial inner membrane localization signal according to the invention has the sequence as set forth in SEQ ID NO: 5. This mitochondrial inner membrane localization signal corresponds to residues 1 to 133 of HTRA2 protein (also known as omi), which allows the precursor form of the protein to be targeted to the mitochondrion and be localized at the inner mitochondrial membrane, facing the intermembrane space (Hermann et al. 2005).

In another embodiment, the mitochondrial inner membrane localization signal is a fragment of SEQ ID NO: 5 capable of allowing the mitochondrial import and attachment of MIA40 to the inner membrane.

In another embodiment, the mitochondrial inner membrane localization signal according to the invention has the sequence as set forth in SEQ ID NO: 25. This mitochondrial inner membrane localization signal corresponds to residues 1 to 105 of Coproporphyrinogen-III oxidase (CPDX) protein, which allows the precursor form of the protein to be targeted to the mitochondrion and be localized at the inner mitochondrial membrane, facing the intermembrane space (Hermann, 2005).

In another embodiment, the mitochondrial inner membrane localization signal is a fragment of SEQ ID NO: 25 capable of allowing the mitochondrial import and attachment of MIA40 to the inner membrane.

The skilled person can readily identify other suitable sequences.

Typically, the mitochondrial inner membrane localization sequence, which allows for the targeting of the polypeptide to the mitochondrial intermembrane space and its association to the inner mitochondrial membrane, can be tested by biochemical techniques that include mitochondrial purification and mitoplast preparation by the selective rupturing of the outer mitochondrial membrane followed by Western blot analyses. Alternatively, the sub-compartmental localization of the recombinant protein can be monitored by fluorescence microscopy-based techniques after a selective permeabilization of the mitochondrial outer membrane using a detergent called digitonin (Otera et al., 2005; Hangen et al, 2010b).

In a preferred embodiment, the polypeptide of the invention comprises:

a) the amino acid sequence as set forth in SEQ ID NO: 4 (MLS of AIF) and
b) the amino acid sequence as set forth in SEQ ID NO: 2 (MIA40 isoform 1).

In a preferred embodiment, the polypeptide of the invention consists in:

a) the amino acid sequence as set forth in SEQ ID NO: 4 (MLS of AIF) fused N-terminally to
b) the amino acid sequence as set forth in SEQ ID NO: 2 (MIA40 isoform 1), with Arginine and Serine amino acids serving as linkers in between SEQ ID NO: 4 and SEQ ID NO: 6.

This polypeptide has the sequence as set forth in SEQ ID NO: 6.

### Nucleic Acids According to the Invention

In one aspect, the invention also relates to a nucleic acid encoding a polypeptide as defined above.

In a preferred embodiment, said nucleic acid has the sequence as set forth in SEQ ID NO: 7. This nucleic acid encodes the polypeptide as set forth in SEQ ID NO: 6.

The following sequences are referred to in the present application:

SEQ ID NO: 1 represents amino acids 28 to 142 of the human MIA40 isoform 1. It contains the oxidase motif and the CX9C—CX9C domain of MIA40.
SEQ ID NO: 2 represents the polypeptide sequence of human MIA40 isoform 1.
SEQ ID NO: 3 represents the polypeptide sequence of human MIA40 isoform 2.
SEQ ID NO: 4 is the N-terminal MLS from AIF.
SEQ ID NO: 5 is the N-terminal MLS from HTRA2 (amino acids 1 to 133 from HTRA2).
SEQ ID NO: 6 is the sequence of a recombinant polypeptide according to the invention: MLS(AIF)-MIA40.
SEQ ID NO: 7 is the nucleic acid sequence encoding SEQ ID NO: 6.
SEQ ID NO: 25 is the N-terminal MLS from CPDX (amino acids 1 to 105 from CPDX).

### Pharmaceutical Compositions and Methods of Treatment of the Invention

The polypeptides and nucleic acids according to the present invention are useful in therapy.

Therefore, the invention also relates to the polypeptides or nucleic acids described above for use as a medicament.

In other words, the invention also relates to the polypeptides or nucleic acids described above for use in a method of treatment.

In particular, the invention relates to polypeptides or nucleic acids described above for use in a method for treating a mitochondrial disorder in a patient.

As used herein, the term “treating” has its general meaning in the art and refers to reversing, alleviating, inhibiting the progress of, or preventing the disorder or condition to which such term applies, or reversing, alleviating, inhibiting the progress of, or preventing one or more symptoms of the disorder or condition to which such term applies.

As used herein, the term “patient” denotes a mammal, such as a rodent, a feline, a canine, and a primate. Preferably, a patient according to the invention is a human.

As used herein, the expression ‘mitochondrial disorder” has its general meaning in the art and refers to any disease caused by or aggravated by impaired mitochondrial function.

Mitochondrial disorders include, but are not limited to, complex I deficiency, myopathic diseases; cardiolipin deficiency; diabetes; obesity; ischemia and/or reperfusion injuries; neurodegenerative diseases such as Parkinson's disease, Alzheimer's disease, Huntington's disease, amyotrophic lateral sclerosis, and retinal affections such as retinal detachment, retinitis pigmentosa and diabetic retinopathy

In a preferred embodiment, said mitochondrial disorder is a complex I deficiency. Complex I deficiency has been observed in various human pathologies, such as Leigh syndrome, hypertrophic cardiomyopathy and encephalomyopathy, macrocephaly, leucodystrophy and myoclonic epilepsy.

In a preferred embodiment, said mitochondrial disorder is selected from the group consisting of Leigh syndrome, hypertrophic cardiomyopathy and encephalomyopathy, macrocephaly, leucodystrophy and myoclonic epilepsy.

In one embodiment, said patient displays a decreased expression of the gene encoding AIF and/or the gene encoding MIA40.

In one embodiment, said patient displays a polymorphism in the AIF gene which alters its activity.

In one embodiment, said patient displays a polymorphism in the MIA40 gene which alters its interaction with AIF and/or its activity.

In a preferred embodiment, said patient displays the rs9839833 polymorphism in the MIA40 gene.

Indeed, it is particularly advantageous to treat such patients with a polypeptide or nucleic acid according to the invention in order to restore the function of MIA40, without a need for a functional interaction with AIF.

In a further aspect, the invention also relates to pharmaceutical compositions comprising the polypeptide or nucleic acid as described above and a pharmaceutically acceptable carrier.

Any therapeutic agent of the invention as above described may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, intraocular, intravenous, intramuscular or subcutaneous administration and the like.

Polypeptides and nucleic acids of the invention may be produced by any technique known per se in the art, such as, without limitation, any chemical, biological, genetic or enzymatic technique, either alone or in combination(s).

Nucleic acids of the invention may be administered to the patient by any method suitable for gene therapy. Typically, the nucleic acid according to the invention may be incorporated into an expression vector, such as a plasmid or a viral vector. Examples of plasmids include replicating plasmids comprising an origin of replication, or integrative plasmids, such as for instance pUC, pcDNA, pBR, and the like. Examples of viral vectors include adenoviral, retroviral, herpes virus and AAV vectors. Such recombinant viruses may be produced by techniques known in the art, such as by transfecting packaging cells or by transient transfection with helper plasmids or viruses.

The polypeptide or variant thereof or the nucleic acid according to the invention may be used in combination with any other therapeutic strategy for treating the mitochondrial disorder.

### Method of Diagnosis According to the Invention

In another aspect, the invention also relates to a method for detecting a decreased expression of the gene encoding AIF and/or the gene encoding MIA40.

This detection is particularly useful in order to identify patients who would benefit most from the method of treatment described above.

This detection step can be carried out by any suitable method known in the art, such as transcriptome and/or proteome-based methods.

Typically, for AIF or MIA40 mRNA expression analyses, total RNA is extracted, reverse transcribed and quantified by real time PCR using gene-specific primers and probes. Protein expression levels can be evaluated by western blot analyses using antibodies directed against AIF and MIA40.

In another aspect, the invention also relates to a method for diagnosing a mitochondrial disorder in a patient comprising the step of analyzing the gene encoding MIA40 in a biological sample obtained from said patient.

Typically, the gene encoding MIA40 is analyzed in order to assess whether the interaction with AIF may be decreased, due to a mutation or polymorphism.

Indeed, the inventors have found that there exist polymorphisms in the MIA40 gene that alter the capacity of the MIA40 protein to interact with AIF, and therefore prevent the MIA40 protein from being correctly targeted to the inner mitochondrial membrane.

In a preferred embodiment, the gene encoding MIA40 is analyzed for the presence of the single nucleotide polymorphism (SNP) in the region encoding amino acids 1 to 27 of the MIA40 isoform 1.

In another embodiment, the gene encoding MIA40 is analyzed for the presence of the single nucleotide polymorphism (SNP) in the region encoding amino acids 1 to 57 of the MIA40 isoform 1.

Indeed, the inventors have shown that amino acids 1 to 27 of SEQ ID NO: 2 are particularly important for the interaction between MIA40 and AIF (minimal interaction domain). They have also shown that the interaction with AIF is further increased for polypeptides comprising amino acids 1 to 57 of MIA40 isoform 1.

The biological sample suitable for carrying out the invention may be a body fluid, such as serum, plasma, blood or urine. It may also be a tissue biopsy of any kind. In a preferred embodiment, the biological sample is serum. Said serum can be freshly collected serum or frozen serum.

Typically, the genomic DNA present in said biological sample is analyzed in order to detect a polymorphism present in the gene encoding MIA40.

For instance, the inventors have shown that the polymorphism identified in the dbSNP database under the number rs9839833 encodes a MIA40 protein in which the amino acid at position 8 is transformed from glycine to tryptophan (hereafter G8W). This G8W polymorphism results in a diminished capacity to interact with AIF.

It is therefore particularly advantageous to treat a patient displaying this polymorphism (and others) in the MIA40 gene with a polypeptide or nucleic acid according to the invention in order to restore the function of MIA40, without a need for interacting with AIF.

In yet another aspect, the invention relates to a method for screening AIF and/or MIA40 variants that alter the interaction between AIF and MIA40.

Typically, variants can be identified by any suitable method known in the art, including, but not limited to, mRNA sequencing, co-immunoprecipitation, pulldown and 2-hybrid.

The pull-down assay is an in vitro method that allows to 1) assess the interaction levels between two proteins and 2) study the effect of the mutations and truncations on each partner protein. In the assay, first the <<bait>> protein (tagged AIF or MIA40; wild type or mutated) is captured on an bead-immobilized tag-specific affinity ligand and then incubated with a protein source (purified recombinant protein or cell lysate) that contains the <<prey>> protein (AIF or MIA40 wild type or mutated). Finally, the associated proteins are eluted from the beads, resolved by gel electrophoresis and analyzed by Western.

In the following, the invention will be illustrated by means of the following examples.

### FIGURE LEGENDS

FIG. 1. Physical interaction between AIF and MIA40. A. Identification of MIA40 as an AIF-binding protein by immunoprecipitation of AIF (or as a control an isotype-matched purified rabbit IgG), followed by the excision of the co-immunoprecipitated protein band (arrow) after 15% SDS/PAGE electrophoresis and mass spectrometry identification of peptides that match (underlined) MIA40. B. Co-immunoprecipitation of endogenous AIF and MIA40 proteins, as performed on human cancer cell lysates. C. Co-immunoprecipitation of endogenous MIA40 with a panel of Flag-tagged C-terminal truncation mutants of AIF. D. Interaction of GST-tagged truncation mutants of MIA40 with AIF contained in the lysate of U2OS cells. The indicated MIA40 derivatives were immobilized on beads, which then were evaluated for their capacity to retain AIF protein. E. Competitive disruption of the interaction between recombinant AIF (retained on glutathione sepharose beads) and recombinant, His-tagged MIA40 protein in the presence of a synthetic peptide corresponding to the N-terminus of MIA40 (peptide 1-27), but not a mutated peptide (peptide 1-27 delta) that lacks the first 6 N-terminal residues. All experiments have been performed at least three times, yielding similar results.

FIG. 2. Impact of AIF and MIA40 depletion on the mitochondrial proteome. U2OS cells were subjected to the transfection with specific siRNAs that deplete AIF (AIFa), MIA40 (MIA40a, MIA40b) or Emerin as a control (Co). Four days later, the abundance of the indicated proteins was measured on triplicate samples by immunoblot (A), and the relative expression levels of proteins were quantified by image analysis (B, C, F). The relative deficiency of complex I and IV proteins (with respect to complex V) was confirmed by measurement of individual respiratory chain complexes using permeabilized cells in an oximeter (D, E). In addition, relative mRNA levels encoding AIF or MIA40 were determined by quantitative RT-PCR, defining the ratio of AIF or MIA40 to three housekeeping genes in control siRNA-transfected cells as 1 (F). Values are means±SEM of triplicates. *p<0.05, **p<0.01, ***p<0.001 calculated by ANOVA followed by Bonferroni post-analyses. Data are representative of at least three experiments that yielded comparable results.

FIG. 3. Kinetic ordering of the AIF/MIA40 pathway. U2OS cells were transfected with the indicated siRNAs for 1 to 4 days, and the abundance of AIF, MIA40 and actin-b was determined by immunoblot (A) and quantified by image analysis (B). To exclude off-target effects, cells were simultaneously transfected with an AIF-specific siRNA (AIFb, which targets the 3′untranslated region [UTR] of the AIF mRNA) and plasmids encoding either of the two AIF iso forms (Flag-tagged, lacking the 3′UTR) or vector only (C). Alternatively (D), cells were transfected with AIF-specific or controls siRNAs, followed by transfection with a plasmid encoding HA-tagged MIA40 (at 48 h), and measurement of de novo expression of the HA-tagged proteins by immunoblot (at 48 h). The kinetic impact of the HQ mutation on AIF, MIA40 and complex-I-20 protein expression was determined in brain samples retrieved at the indicated day post partum. Duplicate samples from wild type (Aif^{+/y}) and mutant (Aif^{hq/y}) mice were analyzed by immunoblot (E) and the relative abundance of each protein was determined by quantitative image analysis (F). Error bars indicate mean values± variance for duplicates. *p<0.05, **p<0.01, ***p<0.001 calculated by ANOVA followed by Bonferroni pot-analyses. These experiments have been repeated twice, yielding similar results.

FIG. 4. Phenotypic reversal of the AIF defect by mitochondrion-targeted MIA40. A. Schematic representation of mitochondrion-targeted MIA40 (MM) and BirA (MB), which both share an identical mitochondrial localization sequence (MLS) derived from AIF1 (residues 1-120). B. Impact of MM and MB on the abundance of mitochondrial proteins of AIF-depleted cells. U2OS cells were stably transfected with either MM or MB and then subjected to the knockdown of emerin (control, Co.) or AIF, using either a siRNA that only targets AIF (AIFc) or another siRNA (AIFa) that targets both AIF and MM or MB (which all contain identical, AIF-derived N-termini). Four days later, the abundance of the indicated proteins was determined. C. Impact of MM and MB on the abundance of mitochondrial proteins in AIF null mutant ES cells. Wild type (Aif^{+/y}) and mutant (Aif^{-/y}) cells were stably transfected with MM or MB. Then lysates were prepared from ES cells (in the presence of LIF) or embryoid bodies (EB10; generated by withdrawal of LIF for 10 days) and analyzed for the expression of transgenic or endogenous proteins. D. Respiratory function of wild type (Aif^{+/y}) or mutant (Aif^{-/y}) ES cells stably transfected with MM or MB. E-F. Phenotype of embryoid bodies derived from wild type (Aif^{+/y}) and mutant (Aif^{-/y}) ES cells stably transfected with MM or MB. Representative pictures are shown for embryoid bodies of the indicated genotype (E). The percentage of cystic EBs (F top panel) and their perimeter was determined (F bottom panel).

FIG. 5. Physical interaction between AIF and MIA40. A. Immunoprecipitation of AIF (or as a control an isotype-matched purified rabbit IgG), revealed by 9% SDS/PAGE electrophoresis. B. Co-immunoprecipitation of endogenous AIF and recombinant HA-tagged MIA40 protein, as performed on human cancer cell lysates. C. Interaction of GST-tagged truncation mutations of AIF with MIA40 contained in the lysate of U2OS cells. The indicated AIF deletion mutants were immobilized on beads, which then were evaluated for their capacity to retain MIA40 protein. D. Interaction of GST-tagged truncation mutations of MIA40 with recombinant AIF. The indicated AIF deletion mutants were immobilized on beads, which then were evaluated for their capacity to retain MIA40 protein. E. Interaction of GST-tagged point mutants of MIA40 with recombinant AIF. The indicated MIA40 mutants (C4S; C53S+C55S; C74S; C87S; C97S) were immobilized on beads, which then were evaluated for their capacity to bind AIF protein. All experiments have been performed at least three times, yielding similar results.

FIG. 6. Impact of AIF and MIA40 depletion on the mitochondrial proteome. A and B. U2OS cells were transfected with specific siRNAs that deplete AIF (AIF a and AIFb), CHCHD3 (CHCHD3), Mia40 (MIA40a, MIA40b, MIA40c) or Emerin as a control (Co). Four days later, the abundance of the indicated proteins was measured on duplicate (A) or single samples (B) by immunoblot (A and B), and the relative expression levels of proteins were quantified by image analysis (C). D. Co-immunoprecipitation of endogenous AIF and MIA40 protein, as performed on human cancer cell lysates. The presence of indicated proteins was checked by immunoblot. Data are representative of at least three experiments that yielded comparable results.

FIG. 7. Kinetic ordering of the AIF/MIA40 pathway. A. U2OS cells were simultaneously transfected with a AIF-specific siRNA (siRNA AIFa) that depletes exclusively the endogenous and the flag-tagged AIF1 and plasmids encoding either of the two AIF isoforms (Flag-tagged AIF1 or AIF2) or vector only. Four days later, the abundance of the indicated proteins was measured on duplicate samples by immunoblot. To exclude off-target effects, cells were simultaneously transfected with an AIF1-specific siRNA (AIFa, which targets only AIF1 mRNA and plasmids encoding either of the two AIF isoforms (Flag-tagged, lacking the 3′UTR) or vector only. B, C. Mouse embryonic stem cell (mES) lines AIF wt (Aif^{+/Y}) and AIF deficient (Aif^{-/Y}) were analyzed by Western blot for the abundance of the indicated proteins (B left) and the relative expression levels of proteins were quantified by image analysis (B right). C. The impact of the HQ mutation on AIF, MIA40 and respiratory chain complexes subunit protein expression was determined on various organs dissected from adult mice. Triplicate samples from wild type (Aif^{y/+}) and mutant (Aif^{y/hq}) mice were analyzed by immunoblot and the relative abundance in each protein was determined by quantitative image analysis (D). In addition, relative mRNA levels encoding AIF or MIA40 were determined by quantitative RT-PCR, defining the ratio of AIF or MIA40 to three housekeeping genes in control organs (E). Error bars indicate mean values± variance for duplicates. *p<0.05, **p<0.01, ***p<0.001 calculated by ANOVA followed by Bonferroni post-analyses. These experiments have been repeated twice, yielding similar results.

FIG. 8. Phenotypic reversion of the AIF defect by mitochondrion-targeted MIA40. A. Impact of MM and MB on the abundance of mitochondrial proteins in AIF-depleted cells. U2OS cells were stably transfected with either MM or the empty vector and then subjected to the knockdown of emerin (control, Co.) or AIF, using either a siRNA that only targets AIF (AIFc) or another siRNA (AIFa) that targets both AIF and MM (which all contain identical, AIF-derived N-termini coding for the mitochondrial localization sequence). Four days later, the abundance of the indicated proteins was determined.

FIG. 9: Physical interaction between AIF and the variant form MIA40 (G8W). A. Schematical representation of GST-tagged MIA40 variants (wt and G8W). B. Interaction of GST-tagged full length (142 residues that comprise all the functional cysteines) and truncation mutant of MIA40 (1-57) with AIF contained in the lysate of U2OS cells. The indicated GST-MIA40 derivatives were immobilized on beads, which then were evaluated for their capacity to retain AIF protein.

FIG. 10: Physical interaction between AIF and MIA40 isoforms. A. Schematical representation of HA-tagged MIA40 isoform 1 variants (wt and G8W) and MIA40 isoform 2. B. Co-immunoprecipitation of HA-tagged full MIA40 iso form 1 variants (wt and G8W) and MIA40 isoform 2 with AIF contained in the lysate of U2OS cells. An anti-HA tag antibody, immobilized on protein G beads, was used for the co-immunoprecipitation of AIF/MIA40 complexes.

FIG. 11. Physical interaction between AIF and MIA40.

A. Interaction of GST-tagged AIF with recombinant His-tagged full length MIA40 in absence or presence of pyridine nucleotides (NAD, NADP, NADH and NADPH). GST-tagged AIF was first immobilized on beads, preincubated with the indicated nucleotide before being evaluated for its capacity to retain MIA40 protein.
B. Interaction of GST-tagged truncation or point mutants of MIA40 with recombinant AIF (103-613), in absence or presence of NADH. The indicated MIA40 derivatives were first immobilized on beads and then evaluated for their capacity to retain AIF (103-613) protein pre-bound or not to NADH.

FIG. 12. Loss of respiratory chain complex CI subunit in MIA40^{-/-} knockout mouse embryos.

A. Schematic representation of the gene trap vector for the construction of mutant MIA40 mice. The retroviral vector (BGEO) that contains a lacZ-neomycin resistance fusion cassette was inserted in the MIA40/CHCHD4 gene.
B. MIA40 invalidation leads to embryonic lethality at gastrulation. Intercrosses of heterozygous mice yielded no MIA40^{-/-} pups out of viable offsprings at birth. In order to determine the time of death of MIA40^{-/-} embryos, litters were examined from E6 to birth. No mutant embryos were ever detected after E8.5. Embryos from E7-8.5 litter were individually staged by somite counting or according to Downs and Davis (Downs et al., 1993), criteria at pre-somitic stages. Mutant embryos were nearly always staged E5.5-6. Heterozygous embryos at E5.5-6 were also recovered from about half the litters containing mutant embryos. Altogether, these data point to an embryonic lethality at the beginning of gastrulation.
C. X-Gal staining of heterozygous embryos from E6 to E9 revealed a widespread expression of MIA40, with the exception of the visceral endoderm at the earliest stages.
D. The impact of MIA40 mutation on respiratory chain complexes subunit protein expression. Extracts of individual embryos, retrieved and staged according to Downs and Davis (Downs et al., 1993), criteria at pre-somitic stages, were analyzed by immunoblot for the relative abundance of each indicated protein using specific antibodies.

FIG. 13: Phenotypic reversal of AIF defect by a mitochondrion-targeted mutant MIA40. Impact of MM (wt and mutcys) on the abundance of mitochondrial proteins of AIF-depleted cells.

U2OS cells were stably transfected with either wt MM or mutcys MM and then subjected to the knockdown of emerin (control, Co.) or AIF, using either a siRNA that only targets AIF (AIFc) or another siRNA (AIFa) that targets both AIF and MM (which contains identical, AIF-derived N-termini). Four days later, the abundance of the indicated proteins was determined.

### EXAMPLES

### Example 1

### MLS-MIA40 can Re-Establish Normal Respiratory Function in AIF-Deficient Cells

### Materials and Methods

Antibodies: The following antibodies were used: anti-actin mouse mAb (CHEMICON, MAB1501); anti-AIF mouse mAb (Santa Cruz, Sc13116); anti-AIF rabbit PAB (Cell Signaling, 4642); anti-MIA40 rabbit PAB (Santa Cruz sc98628); anti-CI SU 20 kDa (NDUFB8) mouse mAb (Mitosciences, MS105); anti-Tim23 mAb (BD Transduction, 611222); polyclonal anti-VDAC rabbit pAb (Cell Signaling, 4866); anti-Hsp60 mouse mAb (Stressgen, SPA-806); anti-total human OXPHOS WB antibody cocktail mouse MAB (Mitosciences; MS601); anti-total rodent OXPHOS WB antibody cocktail mouse MAB (Mitosciences; MS604); anti-Flag M2 mouse mAb (SIGMA, F3165).

Synthetic peptides: Synthetic peptides were prepared by the solid phase method following the Fmoc/tBu chemistry. After cleavage from resin and characterization by LC-MS, they were purified to homogeneity by RP-HPLC and lyophilized.

Plasmids and siRNA: Recombinant plasmids pCMV-AIF1-3×Flag and pCMV-AIF2-3×Flag were constructed as described (Hangen et al., 2010b.) All FLAG-tagged c-terminal deletion mutants of human AIF1 were cloned in the vector pCMV-3×FLAG-CMV-14 (Sigma) using EcoRI and KpnI sites. Indicated AIF deletion mutants were subcloned in the bacterial expression vector pGEX-6P using EcoRI and NotI sites. Recombinant plasmids for the expression of human MIA40 in mammalian cells (pCMV-MIA40-HA) or in bacteria (pT7-His-MIA40 and pT7-GST-MIA40) were obtained from Genocopoeia. Indicated MIA40 deletion mutants were subcloned in the bacterial expression vector pGEX-6P using EcoRI and NotI sites. Cysteine residues present in MIA40 were mutated into serines by using a site-directed mutagenesis kit (QuickChange Site-directed Mutagenesis kit, Stratagene) and primers as described (Hofmann et al., 2005).

The bacterial biotin ligase expression vector 3XHA-BiRA pBUDneo is a kind gift of Dr John Strouboulis (de Boer et al., 2003). For the construction of the plasmid pBUDneo-MLS-BirA (pBUD-MB) the sequence coding for the 3XHA tag was removed from 3XHA-BiRA pBUDneo using NotI and BglII restriction enzymes (New England Biolabs) and replaced by the PCR-amplified N-terminal segment of human AIF1 that codes for the first 120 amino acids. For the construction of pBUDneo-MLS-MIA40 (pBUD-MM), the sequence coding for BirA was removed from pBUDneo-MLS-BirA (pBUD-MB) using BglII and XhoI restriction enzymes (New England biolabs) and replaced by the PCR amplified fragment of cDNA corresponding to the open reading frame of the human MIA40.

Coding sequences for MLS-MIA40 (MM) and MLS-BirA (MB) were PCR-amplified from their respective pBUDneo host vectors using Platinum Taq Polymerase (Invitrogen) and the following primers: 5′-CGTAACTCCCGGGATCCTAAATGTTCCGGTGTGGAGG-3′ (SEQ ID NO: 8) (left) and 5′-CGTAACTGCGGCCGCTTAGGGATAGGCTTACCTTCG-3′ (SEQ ID NO: 9) (right). PCR products were agarose gel-purified using the Geneclean system (MP Biomedicals), and digested with XmaI and NotI restriction enzymes (New England Biolabs). Purified digested products were then introduced in an Epstein-Barr Virus-based episomal vector (kind gift from D. Biard, C E A, France) at AgeI-NotI sites, downstream of the strong and constitutive CAG promoter. The resulting pEBV-MM and pEBV-MB plasmids were sequence-verified to confirm the absence of any mutation in the corresponding coding sequences.

For RNA interference experiments the following siRNA sequences were used: for negative control: Co: CCG UGC UCC UGG GGC UGG G [dT][dT] (SEQ ID NO:10); for human AIF: AIFa: GGG CAA AAU CGA UAA UUC U[dT][dT] (SEQ ID NO:11); AIFb: GCA GAC UUU CUC UGU GUA U[dT][dT] (SEQ ID NO:12); AIFc: GCA UGC UUC UAC GAU AUA [dT][dT] (SEQ ID NO.13); for human MIA40: MIA40a: GCA UGG AUU GAU ACU GCC ATT [dT][dT] (SEQ ID NO:14); MIA40b: GGA AGG AUC GAA UCA UAU UTT[dT][dT] (SEQ ID NO:15); MIA40c: CGA GGA GCA UGG AUU GAU ATT [dT][dT] (SEQ ID NO:16) (Applied Biosystems).

Cell culture and transfection: Human Osteosarcoma U2OS cells (ATCC n° HTB-96) as well as cervix carcinoma Hela cells (ATCC n° CCL-2) were cultured at 37° C. and 5% CO2 in Dulbecco's modified Eagle's medium (DMEM; Gibco) supplemented with 10% heat-inactivated fetal bovine serum (FBS) (PAA) and 1% penicillin/streptomycin. Plasmid and siRNA transfections were performed using in Lipofectamine-2000 reagent (Invitrogen) by following manufacturer's procedure. Pools of stably transfected U2OS cells expressing MLS-MIA40 (MM) or MLS-BirA (MB) were established by selection in the presence of 0.5 mg/ml of Geneticin (Invitrogen).

Western blot analyses: U2OS cells (transfected or not) were lysed at 4° C. for 30 min, in Net.N120 buffer (20 mM Tris-HCl, pH 8.0, 120 mM NaCl, 1 mM EDTA, 0.5% Igepal) supplemented with protease inhibitors (EDTA-free protease inhibitor tablet—Roche Applied Science), and phosphatase inhibitors (PhosSTOP phosphatase inhibitor tablet—Roche Applied Science) in the presence or absence of DNase-free RNAse (10 μg/ml; Roche). The lysate was centrifuged for 10 min at 13,000 g to eliminate cell debris. The proteins present in the supernatant were quantified (Bio-Rad DC protein assay) and subjected to direct Western blot analyses or immunoprecipitation, GST pull down followed by Western blot analysis.

Immunoprecipitation: In brief, protein G-Sepharose CL-4B beads (50 μl of a 50% slurry, GE Healthcare) were coated with the indicated antibodies (2 μg per immunoprecipitation), by incubating for 2 to 3 h at 4° C. in 800 μl of NetN120 buffer (20 mM Tris-HCl, pH 8.0, 120 mM NaCl, 1 mM EDTA, 0.5% Igepal). The antibody-coated beads were washed in NetN120 buffer and incubated for 2 h at 4° C. with the cell lysate, in a final volume of 800 μl. Beads were washed three times with Net.N120 buffer supplemented with protease and phosphatase inhibitors and were then incubated at 4° C. for 30 min, with 50 μl of wash buffer with or without DNase-free RNase (250 μg/ml). After a last wash with NetN120, samples were finally resolved directly by SDS-PAGE (NUPAGE; Invitrogen) after boiling in 1×SB (2% SDS, 10% glycerol, 62.5 mM Tris-HCl, pH 6.8, 100 mM dithiothreitol). After electrophoresis, the gel was subjected either to Western blot analysis or to silver staining procedure (Silver stain plus kit and procedure-Biorad) to visualize co-precipitated protein bands.

Identification of AIF associated proteins by mass spectrometry: Specific protein bands co-purifying with the anti-AIF antibody were excised from the gels, subjected to in-gel tryptic digestion, concentrated with ZipTip μC18 pipette tips (Millipore) and co-eluted directly onto a MALDI target. MALDI-MS and MALDI-MS/MS were performed on an Applied Biosystems 4700 MALDI TOF/TOF. The interpretation of both the MS and MS/MS data was carried out with the GPS Explorer software (Version 3.6, Applied Biosystems). A combined MS peptide fingerprint and MS/MS peptide sequencing search was performed against the NCBI database without taxon restriction using the MASCOT search algorithm. These searches specified trypsin as the digestion enzyme, carbamidomethylation of cysteine as fixed modification, partial oxidation of methionine and phosphorylation of serine, threonine, and tyrosine as variable modifications, and allowed for one missed trypsin cleavage. The monoisotopic precursor ion tolerance was set to 30 ppm and the MS/MS ion tolerance to 0.3 Da. MS/MS peptide spectra with a minimum ion score confidence interval ≧95% were accepted; this was equivalent to a median ion score cut off of approximately 35 in the data set. Protein identifications were accepted with a statistically significant MASCOT protein search score ≧75 that corresponded to an error probability p<0.05 in our data set.

GST pull down: All recombinant pGEX plasmids were used for GST fusion protein production (Kaelin et al 1991). GST fusion proteins were produced in bacteria and recovered on glutathione-Sepharose 4B beads (GE Healthcare). When indicated, after glutathione affinity purification, the GST moiety of the recombinant fusion protein was cleaved off after digestion with the PreScission protease (GE Healthcare) and the pure recombinant AIF (103-613) was recovered in the post-digestion supernatant. For competition experiments, GST-AIF protein was first recovered on glutathione-Sepharose 4B beads, then preincubated with synthetic peptides MIA40 1-27 (MSYCRQEGKDRIIFVTKEDHETPSSAE; SEQ ID NO:17) or MIA40 1-27 delta (EGKDRIIFVTKEDHETPSSAE; SEQ ID NO:18) corresponding respectively to N-terminal residues 1-27 and 7-27 of human MIA40 protein, before being incubated with His-MIA40 recombinant protein.

Tissue extract preparation for immunoblot. Wild type (wt) or Harlequin (Hq) mice were anesthetized and killed by decapitation at the indicated age. All the dissected organs were snap-frozen and then homogenized, using Precellys homogenizer (Bertin), in an ice-cold RIPA 1× buffer, supplemented with protease and phosphatase inhibitors.

RNA Isolation and gene expression analysis by quantitative real-time PCR (qRT-PCR): Total RNA from wild type (wt) or harlequin (Hq) mutant mice organs were extracted using Precellys homogenizer (Bertin) and RNA isolation kit from Qiagen. RNA from human cells in culture were extracted using PARIS kit (Ambion). All RNA samples were then stored at −80° C.

The quantification of RNA samples was achieved using the Nanodrop ND-1000 Spectrophotometer and the integrity of the RNA was verified using the Agilent 2100 Bioanalyzer with the Eukaryote Total RNA Nano assay. One microgram of total RNA was reverse-transcribed in a 20 μl final reaction volume using the High Capacity cDNA Reverse Transcription Kit with RNase inhibitor (Applied Biosystems) following the manufacturer's instructions. For the human and mouse AIF1, the following primers and Taqman MGB probes were custom-made by Applied Biosystems: for human AIF1: forward (hAIF1F): 5′-GGCAAAATCGATAATTCTGTGTTAGTC-3′ (SEQ ID NO: 19); reverse (hAIFR): 5′-CCACCAATTAGCAGGAAAGGAA-3′(SEQ ID NO: 20); probe (hAIFcp): 5′-TGTTTCTGTTCTGGTGTCAG-3′(SEQ ID NO:21); for mouse AIF1: forward (mAIF-F): 5′-CGAGCCCGTGGTATTCGA-3′(SEQ ID NO: 22); reverse (mAIF1R): 5 ‘-CCATTGCTGGAACAAGTTGC-3’(SEQ ID NO: 23); probe (mAIFSc): 5′-ACGGTGCGTGGAAG-3′(SEQ ID NO: 24). TaqMan® probes were labeled with 6-FAM at the 5′ end and with a nonfluorescent MGB quencher at the 3′ end. Each probe was combined with different forward and reverse primers (see list) for AIF1 quantification. TaqMan® gene expression assays for human MIA40 (Hs 01027804_g1), mouse MIA40 (Mm01183497_g1), 18S ribosomal RNA (Hs99999901_s1), Gapdh (Mm99999915_g1), Pgk1 (Mm00435617_m1), Tbp (Mm00446973_m1) were from Applied Biosystems. Quantitative PCR reactions were performed using ABI Prism 7900 HT sequence detection system (Applied Biosystems). Real-time q-PCR amplifications were carried out (10 min 95° C. followed by 45 cycles of 15 sec 95° C. and 1 min 60° C.). Technical replicates were performed for each biological sample. For AIF1 and MIA40 expression quantification, reference genes were selected using the TaqMan® Mouse Endogenous Control Arrays. Briefly, 16 housekeeping genes were tested in triplicate for each sample. The most stable genes were selected by analyzing results with GeNorm and Normfinder functions in Genex 4.3.8 (MultiD, Göteborg, Sweden). The geometric mean of 3 housekeeping genes (Gapdh, Pgk1 and Tbp) was used to normalize gene expression levels of AIF1 and MIA40 for further analysis of q-PCR experiments with the relative quantification method.

Mouse embryonic stem cell culture and transfection: Mouse embryonic stem cell (mES) lines AIF wt (AIF+/Y) and AIF deficient (AIF−/Y) were propagated on 0.1% gelatin (Sigma) coated dishes in mES medium based on High Glucose/Glutamax-I DMEM supplemented with 15% fetal calf serum (Hyclone), 1 mM sodium pyruvate, 100 U/ml penicillin, 100 μg/m1 streptomycin, 1% non-essential amino-acids, 100 μM β-mercaptoethanol (all of them from Invitrogen), and 1000 U/ml leukemia inhibitory factor (ESGro, Millipore). For transfection, 2×104 mES cells per well of a 12-well plate were seeded. The next day, plasmid DNA (pEBV-MB or pEBV-MM) was mixed with FuGENE® HD (Promega) at a 2:1 ratio according to the manufacturer's instructions and 50 μl of complex were added per well. The next day medium was changed and selection with 0.5 μg/ml puromycin (Invitrogen) started 48 hours later. Four stably transfected cell lines expressing respectively MB (MB-AIF+/Y and MB-AIF−/Y) and MM (MM-AIF+/Y and MM-AIF−/Y) were established and then routinely propagated in mES medium supplemented with 0.5 μg/ml puromycin. Medium was changed daily and the cells were grown at 37° C. in atmospheric conditions with additional 5% CO2.

Embryoid body (EB) formation: For the formation of EBs, 2×10⁵ mES cells per well were seeded in 6-well Ultra Low Attachment plates (Corning) in Iscove's modified Dulbecco's medium (Invitrogen) supplemented with 15% fetal calf serum (Hyclone), 2 mM L-glutamin (Invitrogen), 100 U/ml penicillin, 100 μg/ml streptomycin, 50 μg/ml ascorbic acid (Sigma), 450 μM monothioglycerol (Sigma) 200 μg/ml iron-saturated transferrin (Sigma) and 0.3 μg/ml puromycin. Medium was changed every 4 days and plates were incubated at 37° C. in atmospheric conditions supplemented with 5% CO₂. Proportion of cystic EBs was evaluated by morphology.

Assessment of respiratory chain activities: Respiratory chain activities were measured on subconfluent mES (overexpressing MB or MM) or U2OS cells (4 days after knocking-down AIF or MIA40 with the selected siRNA) permeabilized with 0.01% digitonin. Rotenone-sensitive NADH quinone reductase (complex I; EC 1.6.5.3), oligomycin-sensitive ATP hydrolase (complex V; EC 3.6.3.14), cyanide-sensitive cytochrome c oxidase (complex IV; EC 1.9.3.1), malonate-sensitive succinate cytochrome c reductase (an indirect measure of succinate dehydrogenase—EC 1.3.5.1—limiting for this activity; EC 1.3.99.1), glycerol cytochrome c reductase (an indirect measure of glycerol 3 phosphate dehydrogenase,—EC 1.1.5.3—limiting for this activity; antimycin-sensitive quinol cytochrome c reductase (complex III; EC 1.10.2.2), were spectrophotometrically measured at 37° C. using a pseudo dual-wavelength Varian CARY50 spectrophotometer (Victoria, Australia) as described previously (Benit et al., 2006). Results are expressed as means±SEM of activity ratios as these have been established to be the most sensitive markers to detect partial defects in the respiratory chain. All measurements were performed at 37° C. Protein levels were determined by the method of Bradford with bovine serum albumin as a standard. All chemicals were analytical reagent grade from Sigma Chemical Company.

Statistical analyses: GraphPad InStat Software was used for statistical analysis. Groups were compared by one-way ANOVA followed by Bonferroni post analyses. The criterion for statistical significance was set at p<0.05. Data are expressed as mean+SEM.

### Results

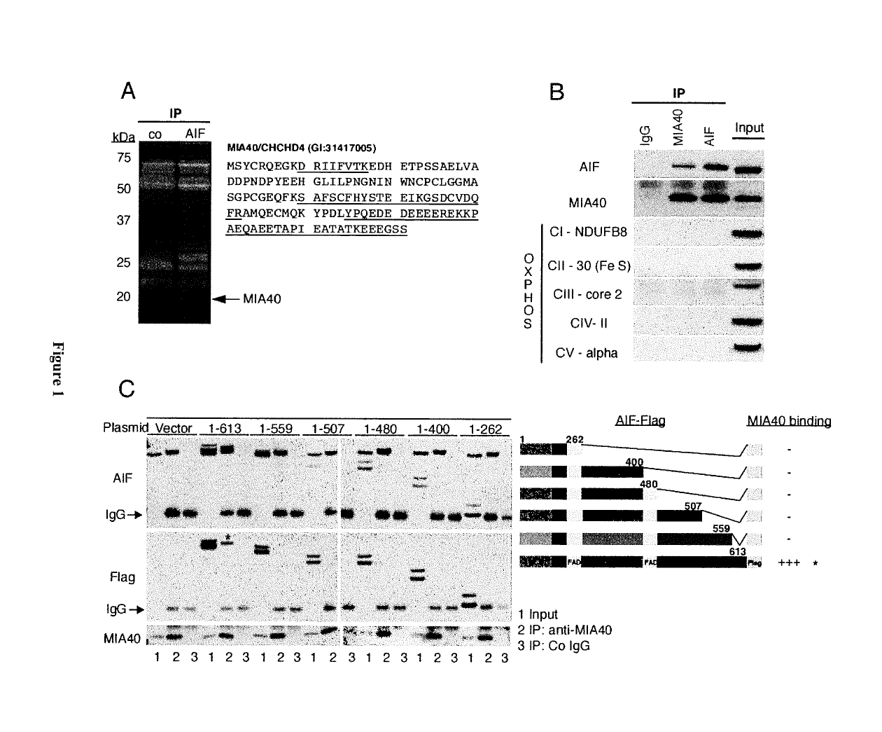

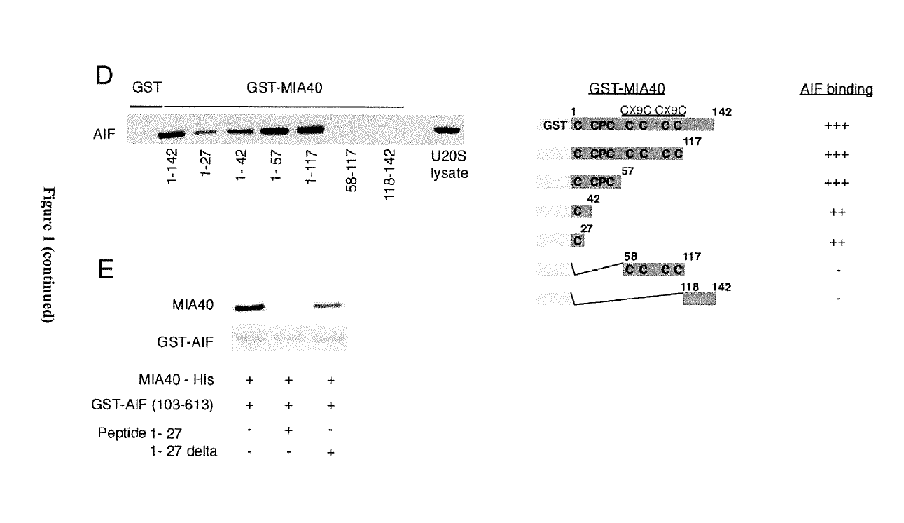

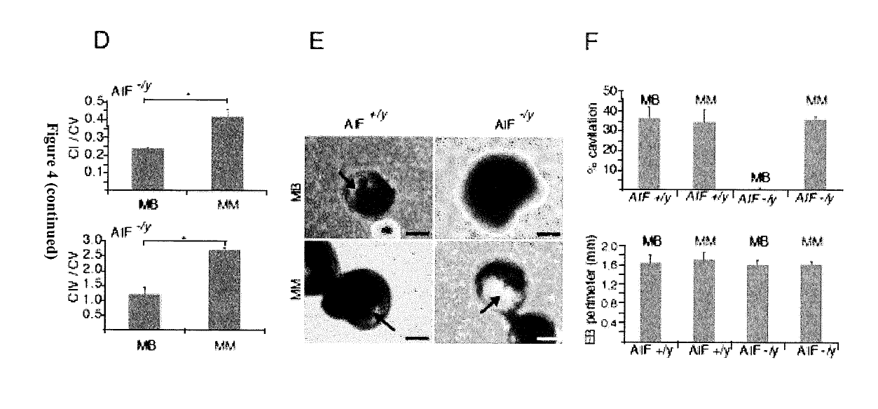

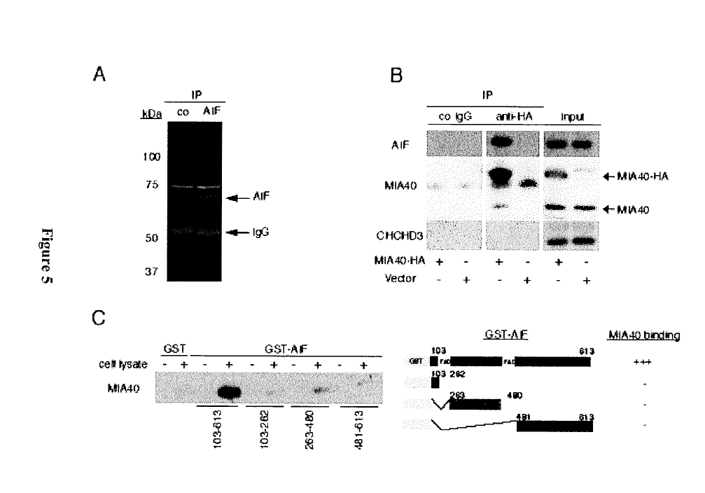

Immunoprecipitation of AIF from human cervical cancer cells, followed by mass spectrometry led to the identification of CHCHD4.1, the human equivalent of yeast Mia40 (Tim40) (Chacinska et al., 2004; Naoe et al., 2004) as an AIF-binding partner (FIG. 1A, B, FIG. 5A).

Human MIA40 is a 16 kDa protein that localizes to the mitochondrial intermembrane space (Hofmann et al., 2005), where it participates in mitochondrial import and catalyzes oxidative protein folding (Chacinska et al., 2008; Banci et al., 2009). Endogenous AIF and MIA40 co-immunoprecipitated in conditions in which AIF failed to interact with other mitochondrial proteins including respiratory chain subunits or CHCHD3 (Schauble et al., 2007), a MIA40 family member (FIG. 1B, FIG. 5B). Only full-length AIF was able to interact with endogenous MIA40 (FIG. 1C, FIG. 5C). Pull down experiments revealed direct interaction of recombinant AIF protein with full-length MIA40 protein, as well as with truncated versions of MIA40 that comprise its N-terminal 27 amino acids (FIG. 1D, FIG. 5D). A synthetic peptide corresponding to this N-terminus was capable of competitively disrupting the interaction of recombinant full-length MIA40 and AIF (FIG. 1E). Mutation of cysteine residues of MIA40, which have been implicated in its mitochondrial accumulation and catalytic activity (Hofmann et al., 2005; Banci et al., 2009), failed to affect binding to AIF (FIG. 5E), suggesting a direct, redox-independent interaction between both proteins.

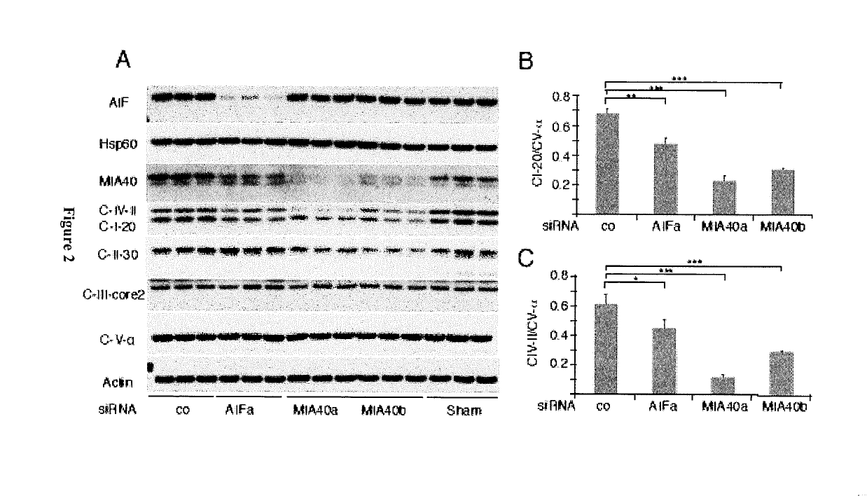

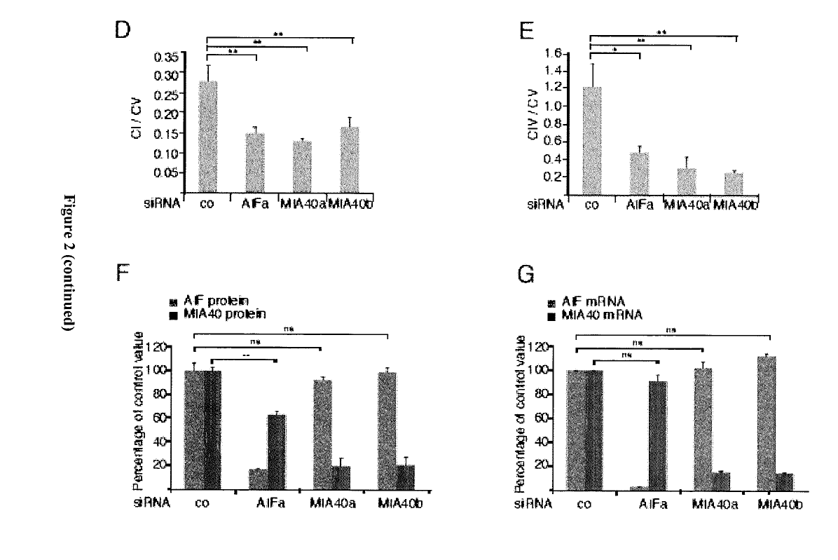

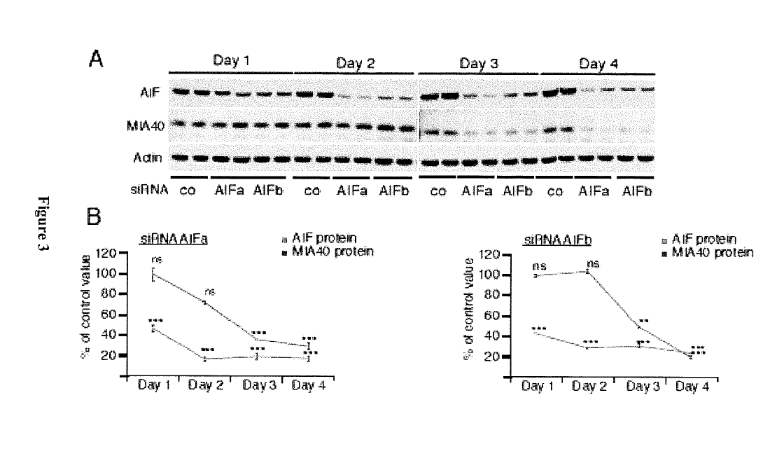

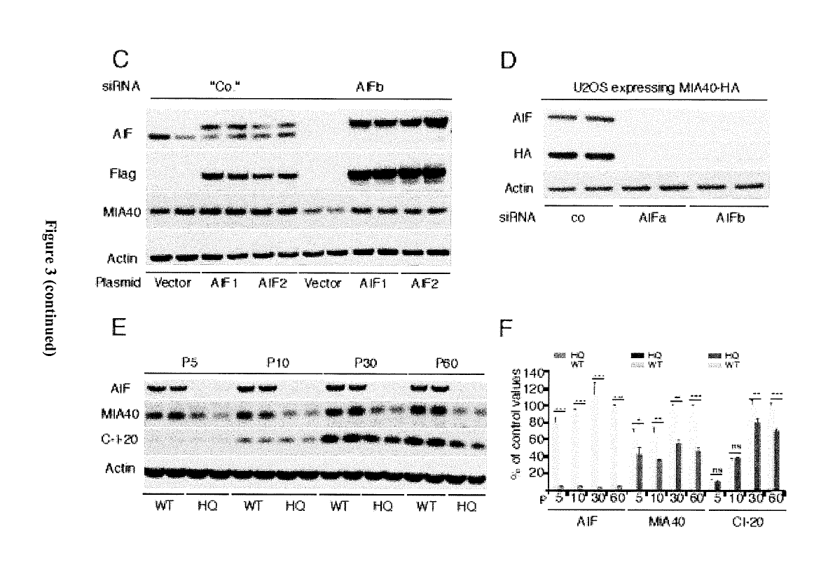

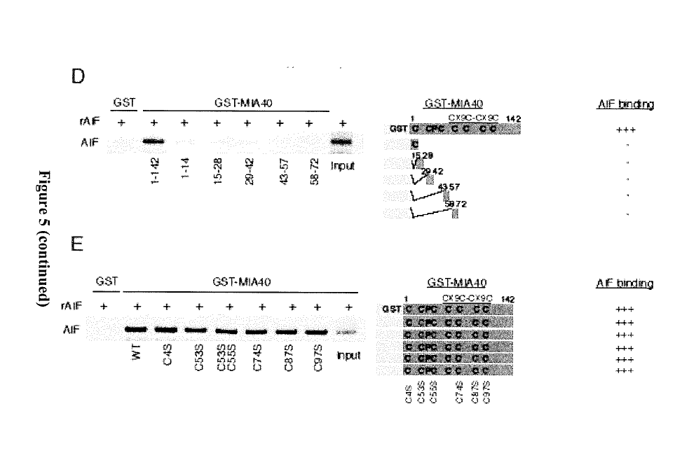

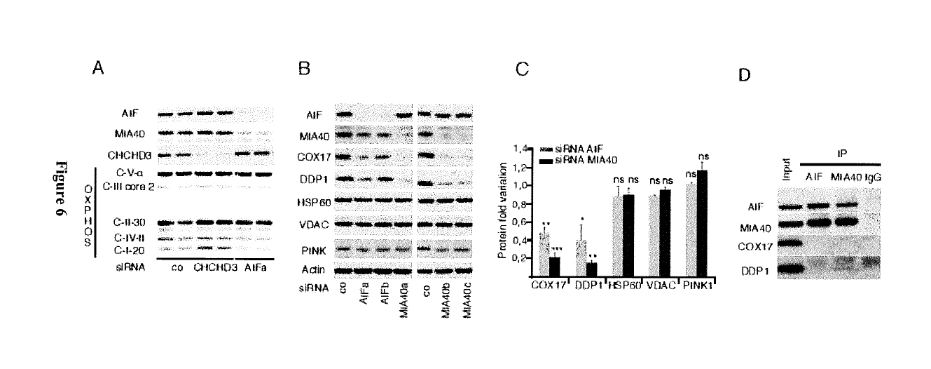

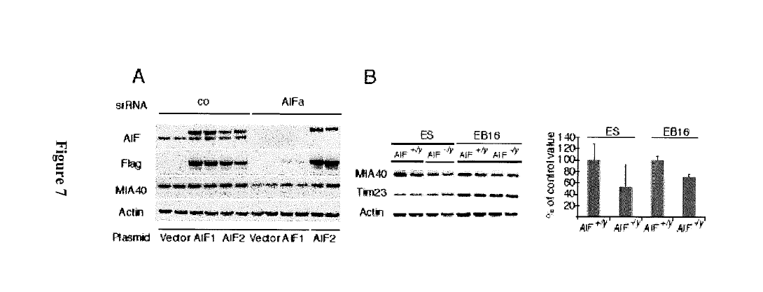

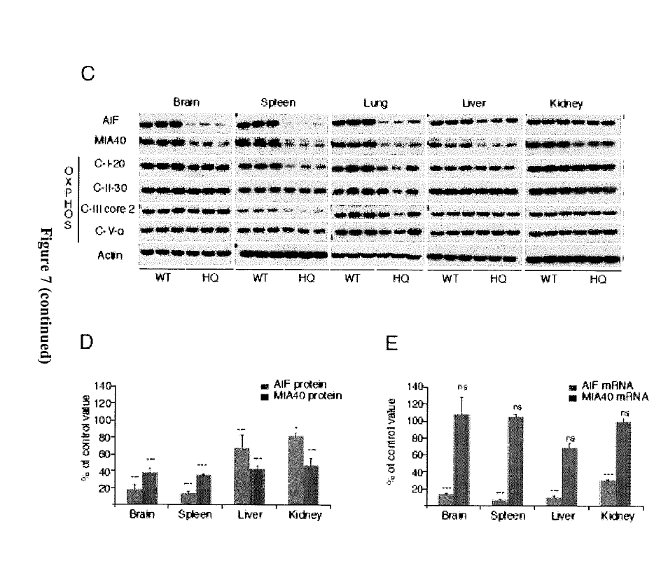

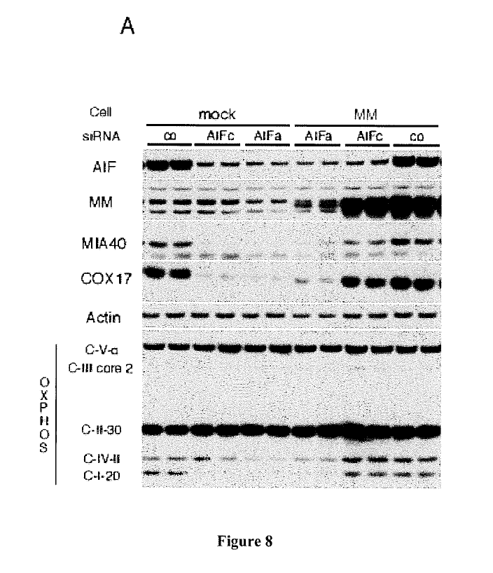

We knocked down MIA40 with two non-overlapping small interfering RNAs (siRNAs). MIA40 depletion resulted in reduction of the protein expression level of several respiratory chain proteins, in particular subunits of complex I and IV (FIG. 2A-C), paralleling a reduced function of complexes I and IV (FIG. 2D,E). As a control, depletion of CHCHD3 failed to mediate similar effects on the stability of analyzed respiratory chain subunits (FIG. 6A). Hence, MIA40 depletion phenocopies the biochemical and functional consequences of AIF depletion at the level of respiratory chain complexes. Accordingly, the knockdown of AIF or MIA40 had similar effects on the abundance of two MIA40 substrates, COX17 and DDP1, yet failed to affect the abundance of other mitochondrial proteins such as VDAC and HSP60 (FIG. 6B, 6C, 6D). The knockdown of MIA40 did not diminish the abundance of AIF (FIG. 2A,F). In stark contrast, depletion of AIF led to a reduction of MIA40 protein expression (FIG. 2A,F) (but not that of CHCHD3 protein, FIG. 6A), without affecting the levels of MIA40-specific mRNA (FIG. 2G). AIF depletion, by two non-overlapping siRNAs, entailed the post-transcriptional reduction of MIA40 protein with a delay of several days (FIG. 3A, B), and re-introduction of AIF expression by means of non-interferable plasmid constructs counteracted the effects of an AIF-specific siRNA on MIA40 expression (FIG. 3C, FIG. 7A). Knockdown of AIF completely abrogated the de novo expression of a HA-tagged MIA40 construct that was cotransfected with AIF-specific siRNAs (FIG. 3D), indicating that AIF is required for the stabilization of MIA40 protein. AIF-deficient (Aif^{-/y}) ES cells and cavitating embryoid bodies (EBs) also manifested reduced MIA40 expression (FIG. 7B). Mice carrying the hypomorphic Aif^{hq/y} mutation manifested a progressive MIA40 defect in the brain post partum that preceded that of complex I subunits during early adulthood, as detected by quantitative immunoblot analyses (FIG. 3E, F). This defect in MIA40 protein expression occurred solely at the post-transcriptional level and was observed also in other organs from Aif^{hq/y} mice (FIG. 7C-E).

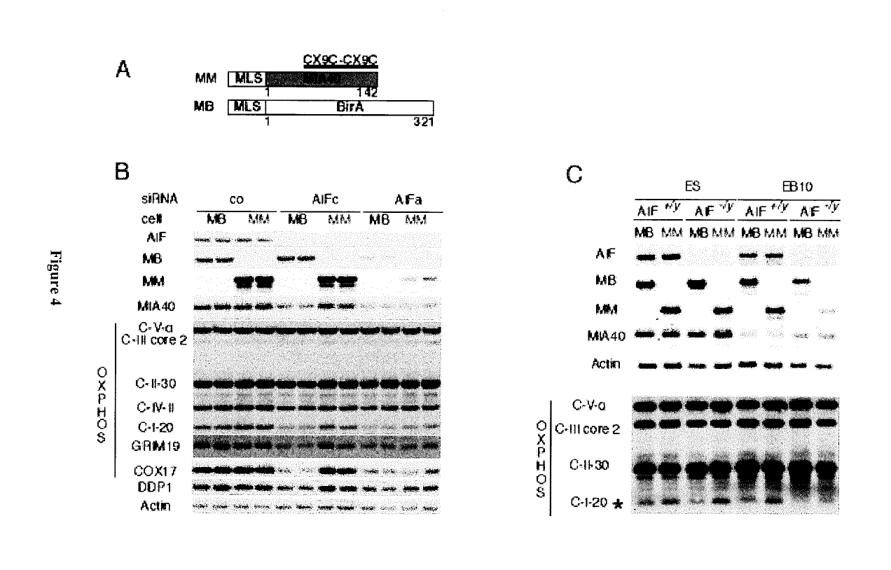

We restored the expression of MIA40 in AIF-depleted cells, by modifying its mitochondrial import pathway and enforcing its attachment to the inner membrane with an appropriate mitochondrial localization sequence (MLS) derived from AIF^{[32]}, fused to its N-terminus (FIG. 4A). The expression level of MLS-MIA40 (MM) was not affected by one of the AIF-specific siRNA (AIFc), yet was strongly reduced by another siRNA (AIFa) that targets the stretch of the mRNA sequence corresponding to the MLS shared by AIF and MM. Importantly, MM was able to revert the defects in DDP1 and respiratory chain complex subunits (C-I-20, GRIM 19 from complex I; C-IV-II and COX17 from complex IV) secondary to AIF depletion (FIG. 4B, 8A). In contrast, a control construct (MB, composed of the MLS from AIF and an irrelevant protein, BirA) was unable to reverse the respiratory chain subunit defect induced by AIF depletion (FIG. 4B). Stable transfection with MM (but not with MB) could also restore the expression of C-I-20 in Aif^{-/y} ES cells that lacked AIF expression (FIG. 4C) as it improved respiratory function (FIG. 4D). Concomitantly, MM (but not MB) restored the capacity of Aif^{-/y} ES cells to form cavitating embryoid bodies (EBs) upon depletion of leukemia-inhibitor factor (LIF), a cytokine that usually maintain the pluripotency and represses the differentiation of ES cells. MM-expressing Aif^{-/y} EBs underwent cavitation upon LIF removal in conditions in which MB-expressing or untransfected (not shown) Aif^{-/y} EBs failed to undergo cavitation (FIG. 4E, F top panel). In contrast, MM had no impact on the formation and size of EBs (FIG. 4F bottom panel). Altogether, these results indicate that restoring MIA40 levels can reverse the phenotype of Aif^{-/y} ES cells with respect to their metabolic and cell death phenotypes.

In conclusion, for the first time our results explain how the absence of AIF can provoke a respiratory defect. Knockdown, knockout or hypomorphic mutation of AIF indistinguishably led to the reduction of the MIA40 protein levels at the post-transcriptional level, linked to a reduced stability of MIA40 protein. We propose that AIF assures the mitochondrial localization of MIA40 through an interaction with its N-terminus, thus stabilizing MIA40 (which, just like other mitochondrial import proteins is probably only stable when present in its orthotopic location). Reduced levels of MIA40 subsequently hamper the correct import and/or triage of protein subunits, mostly of the mitochondrial supercomplex composed by respiratory chain complexes I, III and IV. Reduced respiratory activity then impacts on mitochondrial and cellular metabolism to reduce the propensity of cells to undergo apoptosis, including cavitation-associated cell death. Conversely, restoration of MIA40 levels reverses metabolic and phenotypic alterations driven by AIF deficiency. Altogether, these observations establish a novel link between mitochondrial function and cell death regulation.

The inventors have shown that a polypeptide comprising a MLS fused to MIA40 could bypass the requirement for AIF and restore normal respiratory function.

### Example 2

### Polymorphisms in the MIA40 Gene Disrupt the Interaction with AIF

After having delimited the minimum segment of MIA40 isoform 1 (residues 1-27) required for its interaction with AIF, we found in the public database a polymorphism that targets the same region of MIA40. The identified polymorphism transforms the amino acid glycine at position 8 to a tryptophan (G8W).

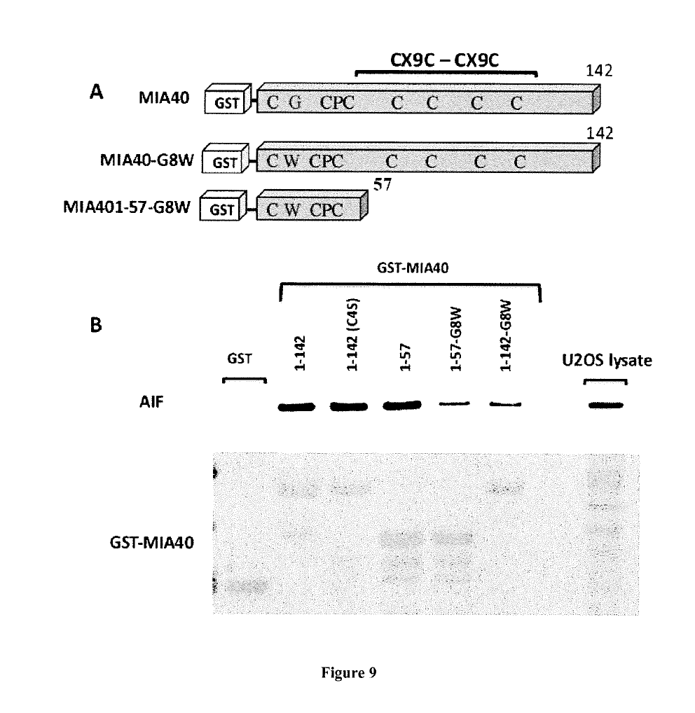

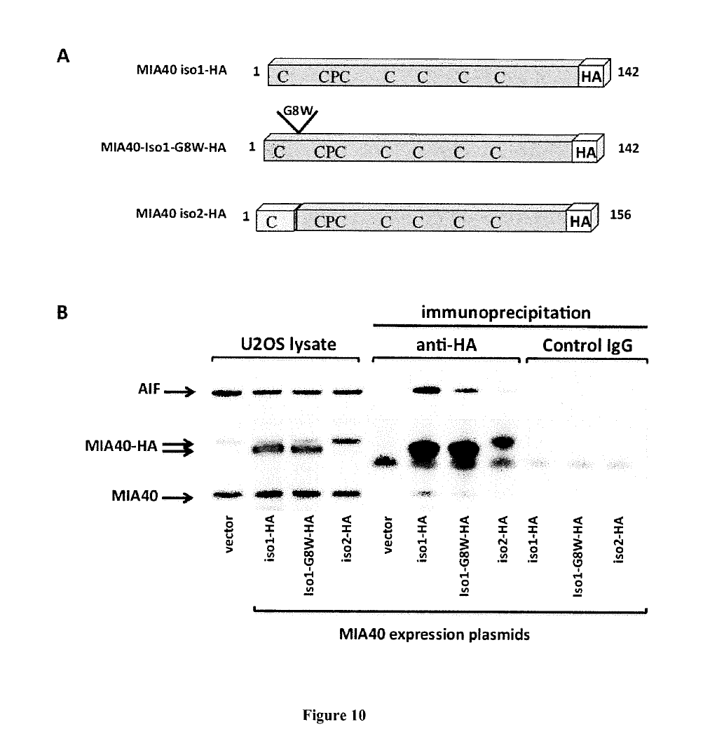

The impact of the G8W polymorphism on the MIA40/AIF complex formation was checked by constructing recombinant plasmids carrying the mutated variant of MIA40 iso form 1. Pull down experiments revealed that compared to the full-length wt MIA40, the variant carrying the G8W polymorphism exhibited a diminished capacity of interaction with AIF (FIG. 9). The same observation was made when the mutation was introduced in a truncated form of MIA40 (MIA40 1-57), which normally binds to AIF with the same efficacy as the full-length protein (FIG. 9). Moreover, a recombinant HA-tagged MIA40 iso form 1 (G8W) variant was overexpressed in a human osteosarcoma derived cell line (U2OS) and checked for its capacity to interact with the endogenous AIF protein. As it is shown in FIG. 9, again compared to the wt version of MIA40, the variant MIA40 (G8W) exhibited a diminished capacity to co-immunoprecipitate with the endogenous AIF (FIG. 10). We conclude that the G8W polymorphism inhibits MIA40 capacity to bind AIF.

### Example 3

### Interaction Between AIF and MIA40 is NADH/NADPH-Dependent

After being imported into the mitochondrion, the mature AIF protein is inserted in the inner membrane facing the inter-membrane space and adopts its final folded configuration through the incorporation of its co-factor FAD (Flavin adenine dinucleotide (Susin et al., 1999). Crystal structure analyses (Ye et al., 2002; Mate et al., 2002) revealed that the flavoprotein AIF bears a similar fold as bacterial nicotinamide adenine dinucleotide (NAD)-dependent oxidoreductases. AIF contains two FAD-binding segments (residues 128 to 262 and 401 to 480) and an NADH binding (residues 263 to 400) that are conserved in non-mammalian AIF orthologs (Modjtahedi et al., 2006; Hangen et al. 2010a). As upon interaction with NADH, AIF undergoes a pronounced conformational change, it is proposed that the formation of AIF/NADH complex must play an important signaling function (Sevrioukova et al., 2009).

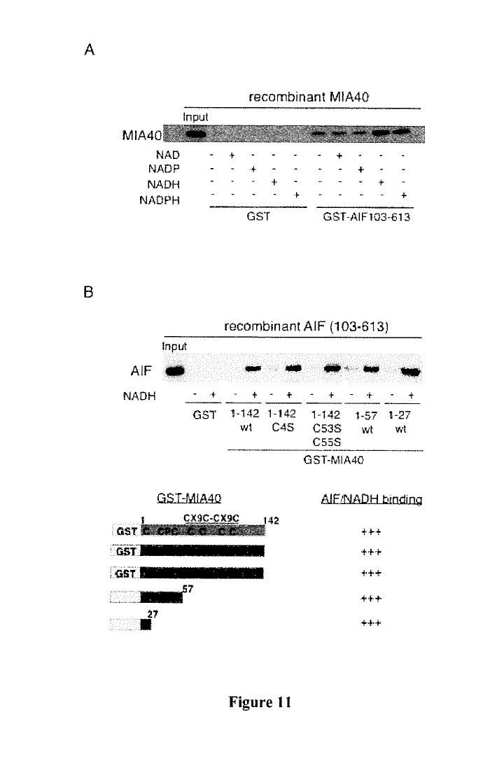

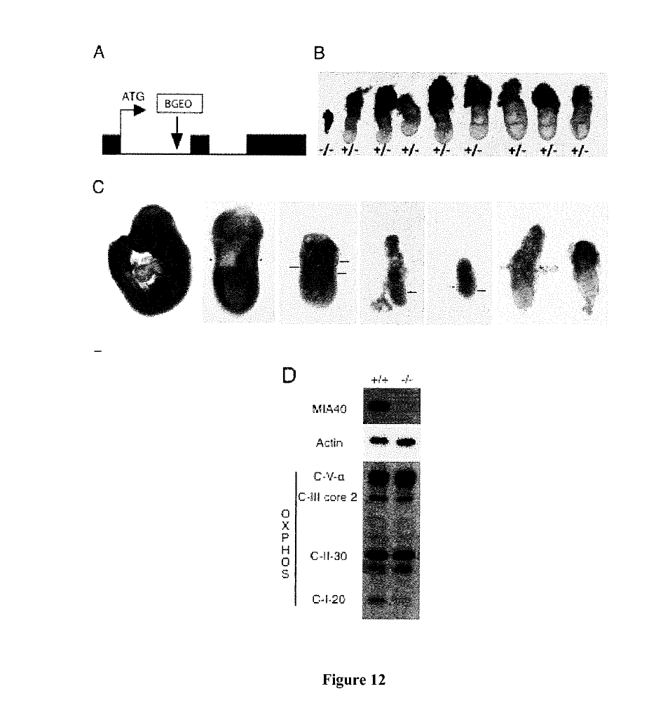

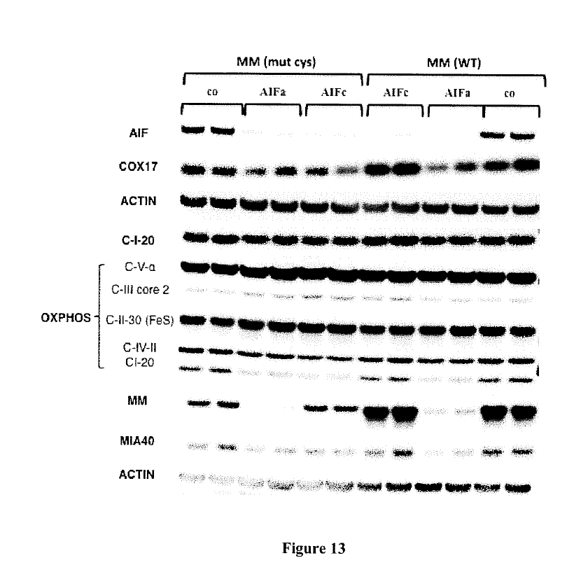

In order to complete the characterization of the physical interaction between AIF and its mitochondrial partner MIA40, the impact of NADH and other pyridine nucleotides (NAD, NADP and NADPH) on the capacity of AIF to interact with MIA40 has been checked. Pull down experiments revealed that the direct interaction of the recombinant AIF protein with full-length MIA40 protein, as well as with truncated versions of MIA40 that comprise its N-terminal 27 amino acids is remarkably enhanced in the presence of NADH and NADPH (FIGS. 11A and 11B). This result indicates that the interaction with its cofactor allows AIF to adopt the optimal conformation that is necessary for the interaction with MIA40.

### Example 4

### Impact of MIA40 Knockout on Respiratory Chain Complex CI Subunit

The knock-down of MIA40 with small interfering RNAs (siRNAs) in cell lines or its downregulation in mice organs carrying the hypomorphic Aif^{hq/y} resulted in reduction of the protein expression level of several respiratory chain proteins, in particular subunits of complex I and IV. The levels of respiratory chain proteins in MIA40^{-/-} knockout mouse embryos that die at gastrulation has been checked (FIG. 12A-C). Intercrosses of heterozygous mice yielded no MIA40^{-/-} pups out of viable offsprings at birth. The time of death of MIA40−/− embryos was nearly always staged around E5.5-6. Western blot analysis of extracts prepared from individual mutant MIA40^{-/-} embryos (FIG. 12D) show that MIA40 depletion in mutant embryos resulted in the reduction of the protein expression level of subunits of complex I and III (FIG. 12D). This observation indicates that, during early embryogenesis, MIA40 plays a crucial role in the regulation of mitochondrial energetics.

### Example 5

### Phenotypic Reversal of the AIF Defect by Mitochondrion-Targeted Mutant MIA40

The expression of MIA40 in AIF-depleted cells was restored by modifying its mitochondrial import pathway and enforcing its attachment to the inner membrane with an appropriate mitochondrial localization sequence (MLS) derived from AIF (Otera et al., 2005), fused to its N-terminus. The stable overexpression of this modified membrane-bound MIA40 protein (MM) restored the expression of C-I-20 in cells in which AIF was knocked down (shown in the previous document). Here, in FIG. 13, it is shown that compared to MM (wt MM), a mutant MM protein (mut cys MM), in which all the functional cysteines of MIA40 (Chacinska et al., 2004, Hofmann S. et al; 2005) were replaced by serines is not anymore able to restore the expression levels of respiratory chain complexes CI and CIV proteins in cells that are knocked down for AIF (FIG. 13). This result points to the importance of MIA40 cysteine motives in the regulation of the observed correction.

### REFERENCES

Throughout this application, various references describe the state of the art to which this invention pertains. The disclosures of these references are hereby incorporated by reference into the present disclosure.
Banci, L., et al., MIA40 is an oxidoreductase that catalyzes oxidative protein folding in mitochondria. Nat Struct Mol Biol, 2009. 16(2): p. 198-206.
Chacinska, A., et al., Essential role of Mia40 in import and assembly of mitochondrial intermembrane space proteins. EMBO J, 2004. 23(19): p. 3735-46.
Chacinska, A., et al., Mitochondrial biogenesis, switching the sorting pathway of the intermembrane space receptor Mia40. J Biol Chem, 2008. 283(44): p. 29723-9.
Claros, M. G. and P. Vincens. Computational method to predict mitochondrially imported proteins and their targeting sequences. Eur J Biochem, 1996, 241(3): p. 779-86.
Claros, M. G. et al. Prediction of N-terminal protein sorting signals. Curr Opin Struct Biol, 1997, 7(3): p. 394-8.
Downs, K. M. and T. Davies, Staging of gastrulating mouse embryos by morphological landmarks in the dissecting microscope. Development, 1993. 118(4): p. 1255-66.
Ghezzi, D., et al., Severe X-linked mitochondrial encephalomyopathy associated with a mutation in Apoptosis-Inducing factor. The American journal of human Genetics, 2010. 86: p. 1-11.
Hangen, E., et al., Life with or without AIF. Trends Biochem Sci, 2010 a. 35(5): p. 278-87.
Hangen, E. et al. A brain-specific isoform of mitochondrial apoptosis-inducing factor: AIF2. Cell Death Differ, 2010 b, 17(7): p. 1155-66.
Herrmann, J. M. and K. Hell. Chopped, trapped or tacked—protein translocation into the IMS of mitochondria. Trends Biochem Sci, 2005, 30(4): p. 205-11.
Hofmann, S., et al., Functional and mutational characterization of human MIA40 acting during import into the mitochondrial intermembrane space. J Mol Biol, 2005. 353(3): p. 517-28.
Joza, N., et al., Muscle-specific loss of apoptosis-inducing factor leads to mitochondrial dysfunction, skeletal muscle atrophy, and dilated cardiomyopathy. Mol Cell Biol, 2005. 25(23): p. 10261-72.
Joza, N., et al., The molecular archaeology of a mitochondrial death effector: AIF in Drosophila. Cell Death Differ, 2008. 15(6): p. 1009-18.
Klein, J. A., et al., The harlequin mouse mutation downregulates apoptosis-inducing factor. Nature, 2002. 419(6905): p. 367-74
Mate, M. J., et al., The crystal structure of the mouse apoptosis-inducing factor AIF. Nat Struct Biol, 2002. 9(6): p. 442-6.
Modjtahedi, N., et al., Apoptosis-inducing factor: vital and lethal. Trends Cell Biol, 2006. 16(5): p. 264-72.
Naoe, M., et al., Identification of Tim40 that mediates protein sorting to the mitochondrial intermembrane space. J Biol Chem, 2004. 279(46): p. 47815-21.
Otera, H., et al., Export of mitochondrial AIF in response to proapoptotic stimuli depends on processing at the intermembrane space. EMBO J, 2005. 24(7): p. 1375-86.
Pospisilik, J. A., et al., Targeted deletion of AIF decreases mitochondrial oxidative phosphorylation and protects from obesity and diabetes. Cell, 2007. 131(3): p. 476-91.
Schauble, S., et al., Identification of ChChd3 as a novel substrate of the cAMP-dependent protein kinase (PKA) using an analog-sensitive catalytic subunit. J Biol Chem, 2007. 282(20): p. 14952-9.
Schmidt, O. et al. Mitochondrial protein import: from proteomics to functional mechanisms. Nat Rev Mol Cell Biol, 2010 11(9): p. 655-67.
Sevrioukova, I. F., Redox-Linked Conformational Dynamics in Apoptosis-Inducing Factor. J Mol Biol, 2009. 390(5): p. 924-38.
Susin, S. A., et al., Molecular characterization of mitochondrial apoptosis-inducing factor. Nature, 1999. 397(6718): p. 441-6.
Vahsen, N., et al., AIF deficiency compromises oxidative phosphorylation. EMBO J, 2004. 23(23): p. 4679-89.
Wissing, S., et al., An AIF orthologue regulates apoptosis in yeast. J Cell Biol, 2004. 166(7): p. 969-74.
Ye, H., et al., DNA binding is required for the apoptogenic action of apoptosis inducing factor. Nat Struct Biol, 2002. 9(9): p. 680-4.

1. A polypeptide comprising:
a) a mitochondrial inner membrane localization signal
and
b) the amino acid sequence as set forth in SEQ ID NO:1 or a variant thereof having at least 80% identity with SEQ ID NO:1.

2. A polypeptide according to claim 1, comprising the amino acid sequence as set forth in SEQ ID NO: 2 or SEQ ID NO: 3 or a variant thereof having at least 80% identity with SEQ ID NO: 2 or 3.

3. A polypeptide according to claim 1, wherein the mitochondrial inner membrane localization signal has the amino acid sequence selected from the group consisting of SEQ ID NO:4, SEQ ID NO:5, SEQ ID NO: 25 and a variant thereof having at least 90% identity with SEQ ID NO:4, SEQ ID NO:5 or SEQ ID NO: 25.

4. A polypeptide according to claim 1, wherein said polypeptide has the sequence as set forth in SEQ ID NO:6.

5. Nucleic acid encoding a polypeptide wherein said polypeptide comprises:
a) a mitochondrial inner membrane localization signal
and
b) the amino acid sequence as set forth in SEQ ID NO:1 or a variant thereof having at least 80% identity with SEQ ID NO:1.

6. Nucleic acid according to claim 5, having the sequence as set forth in SEQ ID NO:7.

7. A method of treating a patient with a mitochondrial disorder comprising
administering to said patient an amount of the polypeptide according to claim 1 or a nucleic acid according to claim 5 sufficient to treat said mitochondrial disorder.

8. (canceled)

9. The method according to claim 7, wherein said mitochondrial disorder is selected from the group consisting of complex I deficiency, myopathic diseases; cardiolipin deficiency; diabetes; obesity; ischemia and/or reperfusion injuries; neurodegenerative diseases, and retinal affections.

10. The method according to claim 9, wherein said complex I deficiency is from the group consisting of Leigh syndrome, hypertrophic cardiomyopathy and encephalomyopathy, macrocephaly, leucodystrophy and myoclonic epilepsy.

11. The method according to claim 7, wherein said patient displays a decreased expression of the gene encoding AIF and/or the gene encoding MIA40.

12. The method according to claim 7, wherein said patient displays a polymorphism in the MIA40 gene which alters its interaction with AIF and/or its activity.

13. The method according to claim 7, wherein said patient displays the rs9839833 polymorphism in the MIA40 gene.

14. A method for detecting a decreased expression of the gene encoding AIF and/or the gene encoding MIA40.

15. A method for diagnosing a mitochondrial disorder in a patient comprising the step of analyzing the gene encoding MIA40 in a biological sample obtained from said patient.

16. The method of claim 9, wherein said neurodegenerative disease is selected from the group consisting of Parkinson's disease, Alzheimer's disease, Huntington's disease, amyotrophic lateral sclerosis.

17. The method of claim 9, wherein said retinal affection is selected from the group consisting of retinal detachment, retinitis pigmentosa and diabetic retinopathy


SEQUENCE LISTING (25 sequences)

SEQ ID NO: 1 - protein, 115 aa, Homo sapiens
LVADDPNDPYEEHGLILPNGNINWNCPCLGGMASGPCGEQFKSAFSCFHYSTEEIKGSDCVDQFRAMQECMQKYPDLYPQEDEDEEEEREKKPAEQAEETAPIEATATKEEEGSS

SEQ ID NO: 2 - protein, 142 aa, Homo sapiens
MSYCRQEGKDRIIFVTKEDHETPSSAELVADDPNDPYEEHGLILPNGNINWNCPCLGGMASGPCGEQFKSAFSCFHYSTEEIKGSDCVDQFRAMQECMQKYPDLYPQEDEDEEEEREKKPAEQAEETAPIEATATKEEEGSS

SEQ ID NO: 3 - protein, 155 aa, Homo sapiens
MPVSYSSVTTRYYHRAGAEEGKDRIIFVTKEDHETPSSAELVADDPNDPYEEHGLILPNGNINWNCPCLGGMASGPCGEQFKSAFSCFHYSTEEIKGSDCVDQFRAMQECMQKYPDLYPQEDEDEEEEREKKPAEQAEETAPIEATATKEEEGSS

SEQ ID NO: 4 - protein, 120 aa, Homo sapiens
MFRCGGLAAGALKQKLVPLVRTVCVRSPRQRNRLPGNLFQRWHVPLELQMTRQMASSGASGGKIDNSVLVLIVGLSTVGAGAYAYKTMKEDEKRYNERISGLGLTPEQKQKKAALSASEG

SEQ ID NO: 5 - protein, 133 aa, Homo sapiens
MAAPRAGRGAGWSLRAWRALGGIRWGRRPRLTPDLRALLTSGTSDPRARVTYGTPSLWARLSVGVTEPRACLTSGTPGPRAQLTAVTPDTRTREASENSGTRSRAWLAVALGAGGAVLLLLWGGGRGPPAVLA

SEQ ID NO: 6 - protein, 264 aa, Homo sapiens
MFRCGGLAAGALKQKLVPLVRTVCVRSPRQRNRLPGNLFQRWHVPLELQMTRQMASSGASGGKIDNSVLVLIVGLSTVGAGAYAYKTMKEDEKRYNERISGLGLTPEQKQKKAALSASEGRSMSYCRQEGKDRIIFVTKEDHETPSSAELVADDPNDPYEEHGLILPNGNINWNCPCLGGMASGPCGEQFKSAFSCFHYSTEEIKGSDCVDQFRAMQECMQKYPDLYPQEDEDEEEEREKKPAEQAEETAPIEATATKEEEGSS

SEQ ID NO: 7 - DNA, 795 bp, Homo sapiens
atgttccggtgtggaggcctggcggcgggtgctttgaagcagaagctggtgcccttggtgcggaccgtgtgcgtccgaagcccgaggcagaggaaccggctcccaggcaacttgttccagcgatggcatgttcctctagaactccagatgacaagacaaatggctagctctggtgcatcagggggcaaaatcgataattctgtgttagtccttattgtgggcttatcaacagtaggagctggtgcctatgcctacaagactatgaaagaggatgaaaaaagatacaatgaaagaatttcagggttagggctgacaccagaacagaaacagaaaaaggccgcgttatctgcttcagaaggaagatccatgtcctattgccggcaggaagggaaggatcgaatcatatttgtaaccaaagaagatcatgaaactccaagcagtgcagaattggtggctgatgaccccaacgatccatacgaggagcatggattgatactgccaaatggaaacattaactggaactgcccatgccttgggggaatggccagcggtccctgtggagaacagtttaagtcagccttttcctgcttccactatagcacggaggagatcaaggggtcagactgtgtagaccagttccgggccatgcaggaatgcatgcagaaatacccagacctctatccccaagaggatgaggatgaggaagaggaaagagagaagaagccagcagaacaagcagaagaaacagctcccattgaggccactgcaaccaaagaagaggagggatcaagttaa

SEQ ID NO: 8 - DNA, 37 bp, Artificial sequence
  Synthetic oligonucleotide primer
cgtaactcccgggatcctaaatgttccggtgtggagg

SEQ ID NO: 9 - DNA, 36 bp, Artificial sequence
  Synthetic oligonucleotide primer
cgtaactgcggccgcttagggataggcttaccttcg

SEQ ID NO: 10 - DNA, 21 bp, Artificial sequence
  Synthetic siRNA
ccgugcuccuggggcugggtt

SEQ ID NO: 11 - DNA, 21 bp, Artificial sequence
  Synthetic siRNA
gggcaaaaucgauaauucutt

SEQ ID NO: 12 - DNA, 21 bp, Artificial sequence
  Synthetic siRNA
gcagacuuucucuguguautt

SEQ ID NO: 13 - DNA, 20 bp, Artificial sequence
  Synthetic siRNA
gcaugcuucuacgauauatt

SEQ ID NO: 14 - DNA, 23 bp, Artificial sequence
  Synthetic siRNA
gcauggauugauacugccatttt

SEQ ID NO: 15 - DNA, 21 bp, Artificial sequence
  Synthetic siRNA
ggaaggaucgaaucauauutt

SEQ ID NO: 16 - DNA, 23 bp, Artificial sequence
  Synthetic siRNA
cgaggagcauggauugauatttt

SEQ ID NO: 17 - protein, 27 aa, homo sapiens
MSYCRQEGKDRIIFVTKEDHETPSSAE

SEQ ID NO: 18 - protein, 21 aa, Homo sapiens
EGKDRIIFVTKEDHETPSSAE

SEQ ID NO: 19 - DNA, 27 bp, Artificial sequence
  Synthetic oligonucleotide primer
ggcaaaatcgataattctgtgttagtc

SEQ ID NO: 20 - DNA, 22 bp, Artificial sequence
  Synthetic oligonucleotide primer
ccaccaattagcaggaaaggaa

SEQ ID NO: 21 - DNA, 20 bp, Artificial sequence
  Synthetic oligonucleotide primer
tgtttctgttctggtgtcag

SEQ ID NO: 22 - DNA, 18 bp, Artificial sequence
  Synthetic oligonucleotide primer
cgagcccgtggtattcga

SEQ ID NO: 23 - DNA, 20 bp, Artificial sequence
  Synthetic oligonucleotide primer
ccattgctggaacaagttgc

SEQ ID NO: 24 - DNA, 14 bp, Artificial sequence
  Synthetic oligonucleotide primer
acggtgcgtggaag

SEQ ID NO: 25 - protein, 105 aa, Homo sapiens
MALQLGRLSSGPCWLVARGGCGGPRAWSQCGGGGLRAWSQRSAAGRVCRPPGPAGTEQSRGLGHGSTSRGGPWVGTGLAAALAGLVGLATAAFGHVQRAEMLPKT